SEQUENCE LISTING (161 sequences)

SEQ ID NO: 1 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVQRLLPVLCQDHG

SEQ ID NO: 2 - protein, 72 aa, Artificial sequence
  synthetic sequence
DSDEHLKKLKTFLENLRRHLDRLDKHIKQLRDILSENPEDERVKDVIDLSERSVRIVKTVIKIFEDSVRKKE

SEQ ID NO: 3 - protein, 74 aa, Artificial sequence
  synthetic sequence
MDDKELDKLLDTLEKILQTATKIIDDANKLLEKLRRSERKDPKVVETYVELLKRHEKAVKELLEIAKTHAKKVE

SEQ ID NO: 4 - protein, 72 aa, Artificial sequence
  synthetic sequence
DSDEHLKKLKKFLENLRRHLDRLKKHIKQLRDILSENPEDKRVKDVIDLSERSVRIVKTVIKIFEDSVRKKE

SEQ ID NO: 5 - protein, 72 aa, Artificial sequence
  synthetic sequence
DSKEHLKKLKKFLENLRRHLDRLKKHIKQLRKILSENPEDKRVKDVIDLSERSVRIVKTVIKIFEDSVRKKE

SEQ ID NO: 6 - protein, 72 aa, Artificial sequence
  synthetic sequence
DSKKHLKKLKKFLENLRRHLDRLKKHIKQLRKILKENPEDKRVKDVIDLSERSVRIVKKVIKIFEDSVRKKE

SEQ ID NO: 7 - protein, 72 aa, Artificial sequence
  synthetic sequence
DSKKHLKKLKKFLENLRRHLDRLKKHIKQLRKILKENPEDKRVKDVIDKSERSVRIVKKVIKIFEDSVRKKE

SEQ ID NO: 8 - protein, 72 aa, Artificial sequence
  synthetic sequence
DSKKHLKKLKKFLENLRRHLDRLKKHIKQLRKILKENPKDKRVKDVIDKSERSVRIVKKVIKIFEKSVRKKE

SEQ ID NO: 9 - protein, 72 aa, Artificial sequence
  synthetic sequence
DSKKHLKKLKKFLENLRRHLDRLKKHIKQLRKILKENPKKKRVKKVIKKSERSVRIVKKVIKIFEKSVRKKE

SEQ ID NO: 10 - protein, 74 aa, Artificial sequence
  synthetic sequence
MKDKELDKLLDTLEKILQKATKIIDDANKLLEKLRRSERKKPKVVETYVELLKRHEKAVKELLEIAKTHAKKVE

SEQ ID NO: 11 - protein, 196 aa, Artificial sequence
  synthetic sequence
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 12 - protein, 75 aa, Artificial sequence
  synthetic sequence
MKDDKELDKLLDTLEKILQTATKIIDKANKLLEKLRRSKRKDPKVVETYVELLKRHEKAVKELLEIAKKHAKKVE

SEQ ID NO: 13 - protein, 74 aa, Artificial sequence
  synthetic sequence
MKDKELDKLLDKLEKILQKATKIIDKANKLLEKLRRSERKKPKVVKTYVELLKRHEKAVKELLEIAKTHAKKVE

SEQ ID NO: 14 - protein, 74 aa, Artificial sequence
  synthetic sequence
MKDKKLDKLLDKLEKILQKATKIIDKANKLLEKLRRSKRKKPKVVKTYVELLKRHEKAVKELLEIAKTHAKKVE

SEQ ID NO: 15 - protein, 75 aa, Artificial sequence
  synthetic sequence
MKKDKKLDKLLDKLEKILQKATKIIDKANKLLEKLRRSKRKKPKVVKTYVELLKRHEKAVKELLEIAKTHAKKVE

SEQ ID NO: 16 - protein, 74 aa, Artificial sequence
  synthetic sequence
MDDKKLDKLLDKLEKILQTATKIIDDANKLLEKLRRSERKDPKVVKTYVELLKRHEKAVKELLEIAKTHAKKVE

SEQ ID NO: 17 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPKQVVAIASXXGGKQALETVQRLLPVLCQDHG

SEQ ID NO: 18 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPRQVVAIASXXGGKQALETVQRLLPVLCQDHG

SEQ ID NO: 19 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIAKXXGGKQALETVQRLLPVLCQDHG

SEQ ID NO: 20 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIARXXGGKQALETVQRLLPVLCQDHG

SEQ ID NO: 21 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVKRLLPVLCQDHG

SEQ ID NO: 22 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVRRLLPVLCQDHG

SEQ ID NO: 23 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVQRLLPVLKQDHG

SEQ ID NO: 24 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVQRLLPVLRQDHG

SEQ ID NO: 25 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVQRLLPVLCQKHG

SEQ ID NO: 26 - protein, 34 aa, Artificial sequence
  synthetic sequence
  MISC_FEATURE (12)..(13): The amino acids at positions 12-13 may be HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means Xaa at position 13 is absent
LTPDQVVAIASXXGGKQALETVQRLLPVLCQRHG

SEQ ID NO: 27 - protein, 35 aa, Artificial sequence
  synthetic sequence
LTPEQVVAIACNKGGKQALKTVQRLLPVLCKPPYC

SEQ ID NO: 28 - protein, 35 aa, Artificial sequence
  synthetic sequence
LTPNQVVAIASNKGGKQALETVQRLLPVLCKPPHR

SEQ ID NO: 29 - protein, 35 aa, Artificial sequence
  synthetic sequence
LTPKQVVAIAGYKGANQALGTVQRLLPVLCKPPYG

SEQ ID NO: 30 - protein, 35 aa, Artificial sequence
  synthetic sequence
LTPKQVVAIANYKGAKQALETVQRLLPLLCKPPYG

SEQ ID NO: 31 - protein, 35 aa, Artificial sequence
  synthetic sequence
LTPKQVVAIASYKGANQALGTVQRLLPVLCKPPYG

SEQ ID NO: 32 - protein, 35 aa, Artificial sequence
  synthetic sequence
MTPKQVVAIASYKGANQALGTVQRLLPVLCKPPYG

SEQ ID NO: 33 - protein, 34 aa, Artificial sequence
  synthetic sequence
LTNDRLVALACIGGRSALNAVKDGLPNALTLIRR

SEQ ID NO: 34 - protein, 35 aa, Artificial sequence
  synthetic sequence
LTPAQVVAIASHNGGKQALKTVQRLLPVLCQAHGL

SEQ ID NO: 35 - protein, 34 aa, Artificial sequence
  synthetic sequence
LVTGQLLKIAKRGGVNAVEAVHASRNALTGAPLH

SEQ ID NO: 36 - protein, 35 aa, Artificial sequence
  synthetic sequence
LTPDQVVAIASNGGGKQALETVRRLLPVLCKPPYR

SEQ ID NO: 37 - protein, 35 aa, Artificial sequence
  synthetic sequence
LTPDQVVAIASNGGGKQALKTVQRLLPVLCKPPYS

SEQ ID NO: 38 - protein, 35 aa, Artificial sequence
  synthetic sequence
LTPNQVVAIASNHGGKQALETVQRLLPVLRKPPYG

SEQ ID NO: 39 - protein, 35 aa, Artificial sequence
  synthetic sequence
LTPEQVVAIASNKGGKQALETVQRLLPVLRKPPYG

SEQ ID NO: 40 - protein, 35 aa, Artificial sequence
  synthetic sequence
LLPHQVVAIVSNSGGKQALETVRRLLPVLCKPPYS

SEQ ID NO: 41 - protein, 34 aa, Artificial sequence
  synthetic sequence
LTPKQVVAIASYGGKQALETVQRLLPVLCKPPYG

SEQ ID NO: 42 - protein, 34 aa, Artificial sequence
  synthetic sequence
LTPKQVVAIASYGGKQSLETVQRLLPVLCKPPYG

SEQ ID NO: 43 - protein, 35 aa, Artificial sequence
  synthetic sequence
LTPKQVVAIASYKGANQALETVQRLLPVLCKPPYG

SEQ ID NO: 44 - protein, 34 aa, Artificial sequence
  synthetic sequence
LTNDRLVALACIGGRSALNAVKDGLPNALTLITR

SEQ ID NO: 45 - protein, 35 aa, Artificial sequence
  synthetic sequence
LTPNQVVAIASGIGGRQALETVHRLLPVLCKPPYG

SEQ ID NO: 46 - protein, 35 aa, Artificial sequence
  synthetic sequence
LTPNQVVAIASHDGGKQALETVQRLLPVLRKPPYG

SEQ ID NO: 47 - protein, 35 aa, Artificial sequence
  synthetic sequence
LTPEQVVAIASHGGAKQALKTVQRLLPVLCQNHGL

SEQ ID NO: 48 - protein, 35 aa, Artificial sequence
  synthetic sequence
LTPEQVVAIASHNGGKQALETVQRLLPVLCKPPYR

SEQ ID NO: 49 - protein, 35 aa, Artificial sequence
  synthetic sequence
LTPKQVVAIASHNGGKQALETVQRLLPVLCHPPYG

SEQ ID NO: 50 - protein, 35 aa, Artificial sequence
  synthetic sequence
LTPKQVVAIASHNGGKQALETVQRLLPVLCQPPYG

SEQ ID NO: 51 - protein, 35 aa, Artificial sequence
  synthetic sequence
LTPNQVVAIASHNGGKQALETVQRLLPVLCKPPYG

SEQ ID NO: 52 - protein, 35 aa, Artificial sequence
  synthetic sequence
LTRNQVVAIASHNGGKQALETVQRLLPVLCKEYGL

SEQ ID NO: 53 - protein, 35 aa, Artificial sequence
  synthetic sequence
LTPEQVVAIASKGGGKQALETVQRLLPVLCKPAYG

SEQ ID NO: 54 - protein, 35 aa, Artificial sequence
  synthetic sequence
LTPNQVVAIASKGGGKQALETVQRLLPVLCQPPYG

SEQ ID NO: 55 - protein, 35 aa, Artificial sequence
  synthetic sequence
LTPDQVVAIASKIGGKQALETVQRLLPVLCKPPYG

SEQ ID NO: 56 - protein, 35 aa, Artificial sequence
  synthetic sequence
LTPAQVVAIASNGGGKQALETVRRLLPVLCQAHGL

SEQ ID NO: 57 - protein, 35 aa, Artificial sequence
  synthetic sequence
LTPARVVAIASNGGGKQALQTVQRLLPVLCEQHGL

SEQ ID NO: 58 - protein, 35 aa, Artificial sequence
  synthetic sequence
LTPDQVVAIASNGGAKQALKTVQRLLPVLCQPPYG

SEQ ID NO: 59 - protein, 35 aa, Artificial sequence
  synthetic sequence
LTPNQVIAIASNGGGKQALETVQRLLPVLCKPPYG

SEQ ID NO: 60 - protein, 35 aa, Artificial sequence
  synthetic sequence
LTPNQVVAIASNHGGKQALETVQRLLPVLCKPPYN

SEQ ID NO: 61 - protein, 35 aa, Artificial sequence
  synthetic sequence
LTPAKVVAIASNIGGKQALETVQRLLPVLCQAHGL

SEQ ID NO: 62 - protein, 35 aa, Artificial sequence
  synthetic sequence
LTPAQVVAIACNIGGKQALETVRRLLPVLCQAHGL

SEQ ID NO: 63 - protein, 35 aa, Artificial sequence
  synthetic sequence
LTPAQVVAIASNIGGKQALETVQRLLPVLCRAHGL

SEQ ID NO: 64 - protein, 35 aa, Artificial sequence
  synthetic sequence
LTPAQVVAIASNIGGKQALETVRRLLPVLCQAHGL

SEQ ID NO: 65 - protein, 35 aa, Artificial sequence
  synthetic sequence
LTPDQVVAIARNIGGKQALETVRRLLPVLCQAHGL

SEQ ID NO: 66 - protein, 35 aa, Artificial sequence
  synthetic sequence
LTPDQVVAIASNIGGKQALKTVQRLLPVLCQAHGL

SEQ ID NO: 67 - protein, 35 aa, Artificial sequence
  synthetic sequence
LTPEQVVTIANNIGGKQALETVQRLLPVLRKPPYG

SEQ ID NO: 68 - protein, 35 aa, Artificial sequence
  synthetic sequence
LTPNQVVTIANNIGGKQALETVQRLLPVLCKPPYG

SEQ ID NO: 69 - protein, 35 aa, Artificial sequence
  synthetic sequence
LTPEQVVAIASNKGGKQALETVQRLLPVLCKPPYG

SEQ ID NO: 70 - protein, 35 aa, Artificial sequence
  synthetic sequence
LTPAQVVAIASNNGGKQALERVQRLLPVLCQAHGL

SEQ ID NO: 71 - protein, 35 aa, Artificial sequence
  synthetic sequence
LTPAQVVAIASNNGGKQALETVRRLLPVLCQAHGL

SEQ ID NO: 72 - protein, 35 aa, Artificial sequence
  synthetic sequence
LTPNQVVAIASNNGAKQALETVQRLLPVLCKPPHP

SEQ ID NO: 73 - protein, 35 aa, Artificial sequence
  synthetic sequence
LTPNQVVAIASNNGGKQALETVQRLLPVLCKPAYG

SEQ ID NO: 74 - protein, 35 aa, Artificial sequence
  synthetic sequence
LTPNQVVAIASNNGGKQALETVQRLLPVLCKPPHP

SEQ ID NO: 75 - protein, 35 aa, Artificial sequence
  synthetic sequence
LTREQVVAIASNNGGKQALETVQRLLPVLRQAHGL

SEQ ID NO: 76 - protein, 35 aa, Artificial sequence
  synthetic sequence
LTRNQVVAIVNNNGGKQALETVHRLLPVLCQPPHG

SEQ ID NO: 77 - protein, 35 aa, Artificial sequence
  synthetic sequence
LTRNQVVAIVNNNGGKQALETVHRLLPVLCQPPYG

SEQ ID NO: 78 - protein, 35 aa, Artificial sequence
  synthetic sequence
LTPAQVVAIASNSGGKQALETVQRLLPVLRQAHGL

SEQ ID NO: 79 - protein, 34 aa, Artificial sequence
  synthetic sequence
LSPNQVVAIASHNGGKPALETVQRLLPVLCKPPY

SEQ ID NO: 80 - protein, 34 aa, Artificial sequence
  synthetic sequence
LLPDQVVAIVSNNGGKLALGTVQRLLPVLCKPPY

SEQ ID NO: 81 - protein, 34 aa, Artificial sequence
  synthetic sequence
LTPAQVVAIASNGGKQALETVRRLLPVLCQAHGL

SEQ ID NO: 82 - protein, 34 aa, Artificial sequence
  synthetic sequence
LTPAQVVAIASNSGGKPALETVRRLLPVLCQAHG

SEQ ID NO: 83 - protein, 34 aa, Artificial sequence
  synthetic sequence
LTPDQVIAIVSNGGGKPALETVRRLLPVLCKHPY

SEQ ID NO: 84 - protein, 34 aa, Artificial sequence
  synthetic sequence
LTPDQVIAIVSNGGGKPALETVRRLLPVLCKPPY

SEQ ID NO: 85 - protein, 34 aa, Artificial sequence
  synthetic sequence
LTPDQVVTIASNNGGKPALETVRRLLPVLCKPPY

SEQ ID NO: 86 - protein, 34 aa, Artificial sequence
  synthetic sequence
LTPNQVVAIASNNGGKPALETVQRLLPVLCKPPY

SEQ ID NO: 87 - protein, 37 aa, Artificial sequence
  synthetic sequence
LTPVQVVAIASNGGKQALATVQRLLPVLCQAHGLAND

SEQ ID NO: 88 - protein, 34 aa, Artificial sequence
  synthetic sequence
LTPKQVVAIASYGGKQALETVQRLLPVLCQPPYG

SEQ ID NO: 89 - protein, 34 aa, Artificial sequence
  synthetic sequence
LSTTRVVSIACIGGRQALKAIKTHMPALRQAPYS

SEQ ID NO: 90 - protein, 34 aa, Artificial sequence
  synthetic sequence
LSTTRVVSIACIGGRQALEAIKTHMPALRQAPYS

SEQ ID NO: 91 - protein, 35 aa, Artificial sequence
  synthetic sequence
LTPQQVVAIASNTGGKQALEAVTVQLRVLRGARYG

SEQ ID NO: 92 - protein, 35 aa, Artificial sequence
  synthetic sequence
LTPQQVVAIASNTGGKRALEAVCVQLPVLRAAPYR

SEQ ID NO: 93 - protein, 35 aa, Artificial sequence
  synthetic sequence
LSTAQVVAVAGRNGGKQALEAVRAQLPALRAAPYG

SEQ ID NO: 94 - protein, 35 aa, Artificial sequence
  synthetic sequence
LSIAQVVAVASRSGGKQALEAVRAQLLALRAAPYG

SEQ ID NO: 95 - protein, 34 aa, Artificial sequence
  synthetic sequence
LSTAQVVAVASGSGGKPALEAVRAQLLALRAAPY

SEQ ID NO: 96 - protein, 35 aa, Artificial sequence
  synthetic sequence
LSTAQVVAVASGSGGKQALEAVRVQLLALRAAPYG

SEQ ID NO: 97 - protein, 35 aa, Artificial sequence
  synthetic sequence
LSTAQVVAVASGSGGKPALEAVRAQLLALRAAPYG

SEQ ID NO: 98 - protein, 35 aa, Artificial sequence; identical to SEQ ID NO: 97
  synthetic sequence

SEQ ID NO: 99 - protein, 35 aa, Artificial sequence
  synthetic sequence
LNTAQVVAIASHDGGKPALEAVRAKLPVLRGVPYA

SEQ ID NO: 100 - protein, 35 aa, Artificial sequence
  synthetic sequence
LSTAQVVAVASHDGGKPALEAVRKQLPVLRGVPHQ

SEQ ID NO: 101 - protein, 35 aa, Artificial sequence; identical to SEQ ID NO: 100
  synthetic sequence

SEQ ID NO: 102 - protein, 35 aa, Artificial sequence
  synthetic sequence
LSTEQVVAIASHNGGKQALEAVKAQLPVLRRAPYG

SEQ ID NO: 103 - protein, 35 aa, Artificial sequence
  synthetic sequence
LSVAQVVTIASHNGGKQALEAVRAQLLALRAAPYG

SEQ ID NO: 104 - protein, 35 aa, Artificial sequence
  synthetic sequence
LNTAQVVAIASHYGGKPALEAVWAKLPVLRGVPYA

SEQ ID NO: 105 - protein, 35 aa, Artificial sequence
  synthetic sequence
LSTAQVVAIASNGGGKQALEGIGEQLRKLRTAPYG

SEQ ID NO: 106 - protein, 35 aa, Artificial sequence
  synthetic sequence
LSPEQVVAIASNHGGKQALEAVRALFRGLRAAPYG

SEQ ID NO: 107 - protein, 35 aa, Artificial sequence
  synthetic sequence
LSTEQVVAIASNHGGKQALEAVRALFRGLRAAPYG

SEQ ID NO: 108 - protein, 35 aa, Artificial sequence
  synthetic sequence
LSTEQVVAIASNKGGKQALEAVKAQLLALRAAPYA

SEQ ID NO: 109 - protein, 35 aa, Artificial sequence
  synthetic sequence
LSTEQVVAIASNNGGKQALEAVKAQLPVLRRAPCG

SEQ ID NO: 110 - protein, 35 aa, Artificial sequence
  synthetic sequence
LSTEQVVAIASNNGGKQALEAVKAQLPVLRRAPYG

SEQ ID NO: 111 - protein, 35 aa, Artificial sequence
  synthetic sequence
LSTEQVVAVASNNGGKQALKAVKAQLLALRAAPYE

SEQ ID NO: 112 - protein, 35 aa, Artificial sequence
  synthetic sequence
LSTAQLVAIASNPGGKQALEAIRALFRELRAAPYA

SEQ ID NO: 113 - protein, 35 aa, Artificial sequence
  synthetic sequence
LSTAQLVAIASNPGGKQALEAVRALFRELRAAPYA

SEQ ID NO: 114 - protein, 35 aa, Artificial sequence
  synthetic sequence
LSTAQLVAIASNPGGKQALEAVRAPFREVRAAPYA

SEQ ID NO: 115 - protein, 35 aa, Artificial sequence
  synthetic sequence
LSTAQLVSIASNPGGKQALEAVRALFRELRAAPYA

SEQ ID NO: 116 - protein, 35 aa, Artificial sequence
  synthetic sequence
LSTAQVVAIASNPGGKQALEAVRALFRELRAAPYA

SEQ ID NO: 117 - protein, 35 aa, Artificial sequence
  synthetic sequence
LTPQQVVAIASNTGGKRALEAVRVQLPVLRAAPYE

SEQ ID NO: 118 - protein, 35 aa, Artificial sequence
  synthetic sequence
LSTAQVVAIATRSGGKQALEAVRAQLLDLRAAPYG

SEQ ID NO: 119 - protein, 35 aa, Artificial sequence
  synthetic sequence
LSTAQVVAIASSHGGKQALEAVRALFRELRAAPYG

SEQ ID NO: 120 - protein, 35 aa, Artificial sequence
  synthetic sequence
LSTAQVATIASSIGGRQALEALKVQLPVLRAAPYG

SEQ ID NO: 121 - protein, 35 aa, Artificial sequence
  synthetic sequence
LSTAQVATIASSIGGRQALEAVKVQLPVLRAAPYG

SEQ ID NO: 122 - protein, 33 aa, Artificial sequence
  synthetic sequence
FRQADIVKIASNGGSAQALNAVIKLGPTLRQRG

SEQ ID NO: 123 - protein, 33 aa, Artificial sequence
  synthetic sequence
FRQADIVKMASNGGSAQALNAVIKLGPTLRQRG

SEQ ID NO: 124 - protein, 33 aa, Artificial sequence
  synthetic sequence
FRQTDIVKMAGSGGSAQALNAVIKHGPTLRQRG

SEQ ID NO: 125 - protein, 33 aa, Artificial sequence
  synthetic sequence
FNRADIVRIAGNGGGAQALYSVRDAGPTLGKRG

SEQ ID NO: 126 - protein, 33 aa, Artificial sequence
  synthetic sequence
FSRADIVRIAGNGGGAQALYSVLDVGPTLGKRG

SEQ ID NO: 127 - protein, 33 aa, Artificial sequence
  synthetic sequence
LQRADIVKIAGNGGGAQALQAVITHRAALTQAG

SEQ ID NO: 128 - protein, 33 aa, Artificial sequence
  synthetic sequence
FSATDIVKIASNIGGAQALQAVISRRAALIQAG

SEQ ID NO: 129 - protein, 33 aa, Artificial sequence
  synthetic sequence
FSAADIVKIASNNGGAQALQAVISRRAALIQAG

SEQ ID NO: 130 - protein, 33 aa, Artificial sequence
  synthetic sequence
FTLTDIVKMAGNNGGAQALKVVLEHGPTLRQRG

SEQ ID NO: 131 - protein, 33 aa, Artificial sequence
  synthetic sequence
FNTEQIVRMVSHDGGSLNLKAVKKYHDALRERK

SEQ ID NO: 132 - protein, 33 aa, Artificial sequence
  synthetic sequence
LDRQQILRIASHDGGSKNIAAVQKFLPKLMNFG

SEQ ID NO: 133 - protein, 33 aa, Artificial sequence
  synthetic sequence
FSAKHIVRIAAHIGGSLNIKAVQQAQQALKELG

SEQ ID NO: 134 - protein, 33 aa, Artificial sequence
  synthetic sequence
LGHKELIKIAARNGGGNNLIAVLSCYAKLKEMG

SEQ ID NO: 135 - protein, 33 aa, Artificial sequence
  synthetic sequence
FNAEQIVRMVSHKGGSKNLALVKEYFPVFSSFH

SEQ ID NO: 136 - protein, 33 aa, Artificial sequence; identical to SEQ ID NO: 135
  synthetic sequence

SEQ ID NO: 137 - protein, 33 aa, Artificial sequence
  synthetic sequence
FNAEQIVSMVSNGGGSLNLKAVKKYHDALKDRG

SEQ ID NO: 138 - protein, 33 aa, Artificial sequence
  synthetic sequence
LEPKDIVSIASHIGATQAITTLLNKWAALRAKG

SEQ ID NO: 139 - protein, 33 aa, Artificial sequence
  synthetic sequence
FNRASIVKIAGNSGGAQALQAVLKHGPTLDERG

SEQ ID NO: 140 - protein, 11 aa, Artificial sequence
  synthetic sequence
GKGSKGKGKGK

SEQ ID NO: 141 - protein, 14 aa, Artificial sequence
  synthetic sequence
GKGSKGKGKGKGSK

SEQ ID NO: 142 - protein, 153 aa, Artificial sequence
  synthetic sequence
MKDKELDKLLDTLEKILQKATKIIDDANKLLEKLRRSERKKPKVVETYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 143 - protein, 153 aa, Artificial sequence
  synthetic sequence
MDDKKLDKLLDKLEKILQTATKIIDDANKLLEKLRRSERKDPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 144 - protein, 154 aa, Artificial sequence
  synthetic sequence
MKDDKELDKLLDTLEKILQTATKIIDKANKLLEKLRRSKRKDPKVVETYVELLKRHEKAVKELLEIAKKHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 145 - protein, 153 aa, Artificial sequence
  synthetic sequence
MKDKELDKLLDKLEKILQKATKIIDKANKLLEKLRRSERKKPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 146 - protein, 153 aa, Artificial sequence
  synthetic sequence
MKDKKLDKLLDKLEKILQKATKIIDKANKLLEKLRRSKRKKPKVVKTYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 147 - protein, 158 aa, Artificial sequence
  synthetic sequence
MKKDKKLDKLLDKLEKILQKATKIIDKANKLLEKLRRSKRKKPKVVKTYVELLKRHEKAVKELLEIAKTHAKKVEGKGSKGKGKGKMDAKSLTAWSRTLVTFKDVFVDFTREEWKLLDTAQQIVYRNVMLENYKNLVSLGYQLTKPDVILRLEKGEEP

SEQ ID NO: 148 - protein, 75 aa, Artificial sequence
  synthetic sequence
PTDEVIEVLKELLRIHRENLRVNEEIVEVNERASRVTDREELERLLRRSNELIKRSRELNEESKKLIEKLERLAT

SEQ ID NO: 149 - protein, 74 aa, Artificial sequence
  synthetic sequence
DNEEIIKEARRVVEEYKKAVDRLEELVRRAENAKHASEKELKDIVREILRISKELNKVSERLIELWERSQERAR

SEQ ID NO: 150 - protein, 72 aa, Artificial sequence
  synthetic sequence
TAEELLEVHKKSDRVTKEHLRVSEEILKVVEVLTRGEVSSEVLKRVLRKLEELTDKLRRVTEEQRRVVEKLN

SEQ ID NO: 151 - protein, 76 aa, Artificial sequence
  synthetic sequence
DLEDLLRRLRRLVDEQRRLVEELERVSRRLEKAVRDNEDERELARLSREHSDIQDKHDKLAREILEVLKRLLERTE

SEQ ID NO: 152 - protein, 72 aa, Artificial sequence
  synthetic sequence
PEDDVVRIIKEDLESNREVLREQKEIHRILELVTRGEVSEEAIDRVLKRQEDLLKKQKESTDKARKVVEERR

SEQ ID NO: 153 - protein, 70 aa, Artificial sequence
  synthetic sequence
DEVRLITEWLKLSEESTRLLKELVELTRLLRNNVPNVEEILREHERISRELERLSRRLKDLADKLERTRR

SEQ ID NO: 154 - protein, 72 aa, Artificial sequence
  synthetic sequence
DEEDHLKKLKTHLEKLERHLKLLEDHAKKLEDILKERPEDSAVKESIDELRRSIELVRESIEIFRQSVEEEE

SEQ ID NO: 155 - protein, 74 aa, Artificial sequence
  synthetic sequence
GDVKELTKILDTLTKILETATKVIKDATKLLEEHRKSDKPDPRLIETHKKLVEEHETLVRQHKELAEEHLKRTR

SEQ ID NO: 156 - protein, 75 aa, Artificial sequence; identical to SEQ ID NO: 15
  synthetic sequence

SEQ ID NO: 157 - protein, 75 aa, Artificial sequence; identical to SEQ ID NO: 15
  synthetic sequence

SEQ ID NO: 158 - protein, 153 aa, Artificial sequence; identical to SEQ ID NO: 146
  synthetic sequence

SEQ ID NO: 159 - protein, 158 aa, Artificial sequence; identical to SEQ ID NO: 147
  synthetic sequence

SEQ ID NO: 160 - protein, 166 aa, Artificial sequence
  synthetic sequence
MGRKKRRQRRRPPQDDKELDKLLDTLEKILQTATKIIDDANKLLEKLRRSERKDPKVVETYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

SEQ ID NO: 161 - protein, 171 aa, Artificial sequence
  synthetic sequence
MRGGRLSYSRRRFSTSTGRDDKELDKLLDTLEKILQTATKIIDDANKLLEKLRRSERKDPKVVETYVELLKRHEKAVKELLEIAKTHAKKVEGSGGGGGMDAKSLTAWSRTLVTFKDVFVDFTREEWKLLDTAQQIVYRNVMLENYKNLVSLGYQLTKPDVILRLEKGEEP

(19) United States
(12) Patent Application Publication
(10) Pub. No.: US 2022/0372089 A1
(43) Pub. Date: Nov. 24, 2022

(54) CELL PERMEABLE PROTEINS FOR GENOME ENGINEERING

(71) Applicant: Altius Institute for Biomedical Sciences, Seattle, WA (US)

(72) Inventors: ALEXANDER J. FEDERATION, Seattle, WA (US); JOHN A. STAMATOYANNOPOULOS, Seattle, WA (US)

(21) Appl. No.: 17/605,995

(22) PCT Filed: Apr. 23, 2020
(86) PCT No.: PCT/US20/29488

§ 371 Date: Oct. 22, 2021

Related U.S. Application Data
(60) Provisional application No. 62/838,583, filed on Apr. 25, 2019.

(51) Int. Cl. C07K 14/47; C12N 9/02; C12N 9/22

(52) U.S. Cl. CPC C07K 14/4702; C12N 9/0071; C12N 9/22

(57) ABSTRACT

The present disclosure provides genome engineering proteins, e.g., nucleic acid binding domains and/or functional domains that have a net positive charge and are cell permeable and can be introduced into the cells without the use of a carrier such as micelles, vesicles, liposomes, and the like.

### CROSS REFERENCE TO RELATED APPLICATION

Pursuant to 35 U.S.C. § 119(e), this application claims priority to the filing date of U.S. provisional application Ser. No. 62/838,583, filed Apr. 25, 2019, the disclosure of which is herein incorporated by reference.

### INCORPORATION BY REFERENCE OF SEQUENCE LISTING

A Sequence Listing is provided herewith as a text file, “ALTI-726WO Seq List_ST25.txt,” created on Apr. 23, 2020 and having a size of 88 KB. The contents of the text file are incorporated by reference herein in their entirety.

### INTRODUCTION

Genome engineering involves genome editing and gene regulation techniques which use nucleic acid binding domains that bind to a target nucleic acid. The nucleic acid binding domains are associated with (e.g., via fusion or interaction) functional domains that mediate genome editing or gene regulation. Nucleic acid binding domains and functional domains, if provided separately, can be introduced into cells as nucleic acids or proteins.

Introduction of proteins for genome engineering offers many advantages over introduction of nucleic acids. However, introduction of proteins into cells requires use of micelles, liposomes and other vehicles to transport the proteins across the cell membrane. Therefore, there is a need for cell permeable genome engineering proteins.

### SUMMARY

The present disclosure provides genome engineering proteins, e.g., nucleic acid binding domains, that are cell permeable and can be introduced into the cells without the use of a carrier such as micelles, vesicles, liposomes, and the like.

In certain aspects, the genome engineering proteins have an overall positive charge. The overall positive charge is obtained by using nucleic acid binding domains (NBD, e.g., DNA binding domain, DBD) that include repeat units that mediate binding to a base in a nucleic acid, which repeat units are naturally occurring and have been identified as having a net positive charge of at least +2 or which repeat units have been modified by substituting neutral or negatively charged amino acids with positively charged amino acids, such that the repeat unit has a net positive charge of at least +2.

In certain aspects, instead of or in addition to modifying the amino acid sequence of a genome engineering protein, a fusion partner is conjugated to the genome engineering protein, which fusion partner has an overall positive charge thereby rendering the conjugated genome engineering protein cell permeable.

### BRIEF DESCRIPTION OF THE DRAWINGS

FIG. 1A. TALEN protein rendered positive by conjugating a cysteine in each repeat with Arg₉ peptide. FIG. 1B. TALEN protein pair transported into a cell as positively charged proteins (via conjugation to Arg₉ peptide) mediated genome editing at a level comparable to editing achieved by introduction of the TALEN pair by transfection of RNA encoding the TALEN pair.

FIG. 2. Heterodimer pairs for conjugation with a nucleic acid binding domain and a function domain. Amino acid residues unlikely to mediate formation of dimer are indicated by rectangles.

FIG. 3. KRAB rendered cell permeable by fusion to a positively charged first member of a heterodimer pair is transported across cell membrane and targeted to TIM3 gene promoter bound by DNA binding domain fused to a second member of the dimer pair.

### DETAILED DESCRIPTION

The present disclosure provides genome engineering proteins, e.g., nucleic acid binding domains, that are cell permeable and can be introduced into the cells without the use of a carrier such as micelles, vesicles, liposomes, and the like.

In certain aspects, the genome engineering proteins have been rendered cell permeable by modifying their amino acid sequence such that the proteins have an overall positive charge.

In certain aspects, instead of or in addition to modifying the amino acid sequence of a genome engineering protein, a fusion partner is conjugated to the genome engineering protein, which fusion partner has an overall positive charge thereby rendering the conjugated genome engineering protein cell permeable.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a protein” includes a plurality of such proteins and reference to “the polynucleotide” includes reference to one or more polynucleotides, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

As used herein, the term “derived” in the context of a polypeptide refers to a polypeptide that has a sequence that is based on that of a protein from a particular source (e.g., an animal pathogen such as Legionella). A polypeptide derived from a protein from a particular source may be a variant of the protein from the particular source (e.g., an animal pathogen such as Legionella). For example, a polypeptide derived from a protein from a particular source may have a sequence that is modified with respect to the protein's sequence from which it is derived. A polypeptide derived from a protein from a particular source shares at least 30% sequence identity with, at least 40% sequence identity with, at least 50% sequence identity with, at least 60% sequence identity with, at least 70% sequence identity with, at least 80% sequence identity with, or at least 90% sequence identity with the protein from which it is derived.

The term “modular” as used herein in the context of a nucleic acid binding domain, e.g., a modular animal pathogen derived nucleic acid binding domain (MAP-NBD) indicates that the plurality of repeat units present in the NBD can be rearranged and/or replaced with other repeat units and can be arranged in an order such that the NBD binds to the target nucleic acid. For example, any repeat unit in a modular nucleic acid binding domain can be switched with a different repeat unit. In some embodiments, modularity of the nucleic acid binding domains disclosed herein allows for switching the target nucleic acid base for a particular repeat unit by simply switching it out for another repeat unit. In some embodiments, modularity of the nucleic acid binding domains disclosed herein allows for swapping out a particular repeat unit for another repeat unit to increase the affinity of the repeat unit for a particular target nucleic acid. Overall, the modular nature of the nucleic acid binding domains disclosed herein enables the development of genome editing complexes that can precisely target any nucleic acid sequence of interest.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific embodiments, the terms refer to a polymeric form of amino acids of any length which include genetically coded amino acids. In particular embodiments, the terms refer to a polymeric form of amino acids of any length which include genetically coded amino acids fused to a heterologous amino acid sequence.

The term “heterologous” refers to two components that are defined by structures derived from different sources. For example, in the context of a polypeptide, a “heterologous” polypeptide may include operably linked amino acid sequences that are derived from different polypeptides (e.g., a NBD and a functional domain derived from different sources). Similarly, in the context of a polynucleotide encoding a chimeric polypeptide, a “heterologous” polynucleotide may include operably linked nucleic acid sequences that can be derived from different genes. Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) may be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

As used herein, the term “cleavage” refers to the breakage of the covalent backbone of a nucleic acid, e.g., a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, the polypeptides provided herein are used for targeted double-stranded DNA cleavage.

A “cleavage half-domain” is a polypeptide sequence which, in conjunction with a second polypeptide (either identical or different) forms a complex having cleavage activity (preferably double-strand cleavage activity).

A “target nucleic acid,” “target sequence,” or “target site” is a nucleic acid sequence that defines a portion of a nucleic acid to which a binding molecule, such as, the NBD disclosed herein will bind. The target nucleic acid may be present in an isolated form or inside a cell. A target nucleic acid may be present in a region of interest. A “region of interest” may be any region of cellular chromatin, such as, for example, a gene or a non-coding sequence within or adjacent to a gene, in which it is desirable to bind an exogenous molecule. Binding can be for the purposes of targeted DNA cleavage and/or targeted recombination, targeted activated or repression. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, promoter sequences, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

An “exogenous” molecule is a molecule that is not normally present in a cell but can be introduced into a cell by one or more genetic, biochemical or other methods. An exogenous molecule can comprise, for example, a functioning version of a malfunctioning endogenous molecule, e.g. a gene or a gene segment lacking a mutation present in the endogenous gene. An exogenous nucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” of gene expression refers to a change in the activity of a gene. Modulation of expression can include, but is not limited to, gene activation and gene repression. Genome editing (e.g., cleavage, alteration, inactivation, donor integration, random mutation) can be used to modulate expression. Gene inactivation refers to any reduction in gene expression as compared to a cell that does not include a polypeptide or has not been modified by a polypeptide as described herein. Thus, gene inactivation may be partial or complete.

The terms “patient” or “subject” are used interchangeably to refer to a human or a non-human animal (e.g., a mammal).

The terms “treat”, “treating”, treatment” and the like refer to a course of action (such as administering a polypeptide comprising a NBD fused to a heterologous functional domain or a nucleic acid encoding the polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action (such as administering a polypeptide comprising a NBD fused to a heterologous functional domain or a nucleic acid encoding the polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

The terms “conjugating,” “conjugated,” and “conjugation” refer to an association of two entities, for example, of two molecules such as two proteins, two domains (e.g., a binding domain and a cleavage domain), or a protein and an agent, e.g., a protein binding domain and a small molecule. The association can be, for example, via a direct or indirect (e.g., via a linker) covalent linkage or via non-covalent interactions. In some embodiments, the association is covalent. In some embodiments, two molecules are conjugated via a linker connecting both molecules. For example, in some embodiments where two proteins are conjugated to each other, e.g., a binding domain and a cleavage domain of an engineered nuclease, to form a protein fusion, the two proteins may be conjugated via a polypeptide linker, e.g., an amino acid sequence connecting the C-terminus of one protein to the N-terminus of the other protein. Such conjugated proteins may be expressed as a fusion protein.

The term “consensus sequence,” as used herein in the context of nucleic acid or amino acid sequences, refers to a sequence representing the most frequent nucleotide/amino acid residues found at each position in a plurality of similar sequences. Typically, a consensus sequence is determined by sequence alignment in which similar sequences are compared to each other. A consensus sequence of a protein can provide guidance as to which residues can be substituted without significantly affecting the function of the protein.

As used herein, the term “genome modifying proteins” refer to nucleic acid binding domains and functional domains which cooperate to modify genome or epigenome is a cell. Examples of genome modifying proteins are provided herein and include but are not limited to nucleic acid binding proteins comprising modular repeat units, nucleic acid binding proteins comprising zinc fingers, functional domains such as labels, tags, polypeptides having nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, e.g., nucleases, transcriptional activators, transcriptional repressors, chromatin modifying protein, and the like. Genome modifying proteins also encompass a single polypeptide comprising a nucleic acid binding domain and functional domain or two or more polypeptides, where a first polypeptide comprises a nucleic acid binding domain and a second polypeptide comprises a functional domain and wherein the first and second polypeptide associate with each other via a non-covalent interaction, such as, via a interactions mediated by first and second members of a heterodimer, where one of the first and second polypeptide is conjugated to the first member and the other polypeptide is conjugated to the second member. Such heterodimers are provided herein.

As used herein the terms “overall charge” or “net charge” refers to the theoretical charge of a protein at physiological pH based upon its amino acid sequence. In certain aspects, the amino acid substitutions disclosed herein may increase the theoretical net charge (at physiological pH) of the polypeptide being modified by at least +1, +2, +3, +4, +5, +10, +15, or more.

As used herein, a “fusion protein” includes a first protein moiety, e.g., a nucleic acid binding domain, having a peptide linkage with a second protein moiety. In certain aspects, the fusion protein is encoded by a single fusion gene.

### Positively Charged Genome and Epigenome Modifying Proteins

As set forth above, genome engineering proteins that are cell permeable and can be introduced into the cells without the use of a carrier such as micelles, vesicles, liposomes, and the like are disclosed herein. The genome engineering proteins have been rendered cell permeable by making the proteins positively charged as explained below.

### Positively Charged Nucleic Acid Binding Domains

The present disclosure provides a genome engineering protein that may be a polypeptide comprising a nucleic acid binding domain (NBD) comprising at least one repeat unit (RU) comprising a 33-36 amino acid long sequence having at least 80% sequence identity to the amino acid sequence:

(SEQ ID NO: 1)
LTPDQ VVAIA SX¹²X¹³GG KQALE TVQRL LPVLC QDHG,

or having the sequence of SEQ ID NO:1 with one or more conservative amino acid substitutions thereto; and comprising at least one of the following amino acid substitutions relative to SEQ ID NO:1: D4K/R/H; S11K/R/H; Q23K/R/H; C30K/R/H; and D32K/R/H, wherein X₁₂X₁₃ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent, wherein when the repeat unit comprises the substitution:

i) D4K, X¹²X¹³ is not HN, YK or YG or wherein when the repeat unit comprises the substitution D4K, the repeat unit further comprises at least one of the following substitutions S11K/R/H; Q23K/R/H; C30K/R/H; and D32K/R/H,

ii) S11K, X¹²X¹³ is not RG or NI, or wherein when the repeat unit comprises the substitution S11K, the repeat unit further comprises at least one of the following substitutions D4K/R/H; Q23K/R/H; C30K/R/H; and D32K/R/H,

iii) Q23K, X¹²X¹³ is not SI, CI, or NN, wherein when the repeat unit comprises the substitution Q23R, X¹²X¹³ is not NG, or the repeat unit further comprises at least one of the following substitutions D4K/R/H; S11K/R/H; C30K/R/H; and D32K/R/H,

iv) C30R, X¹²X¹³ is not NS, HD, NI, NN, NH or NK, or the repeat unit further comprises at least one of the following substitutions D4K/R/H; S11K/R/H; Q23K/R/H; and D32K/R/H,

v) D32H, X¹²X¹³ is not NG, or the repeat unit further comprises at least one of the following substitutions D4K/R/H; S11K/R/H; Q23K/R/H; and C30K/R/H, and

wherein the repeat unit has a theoretical net charge of at least +2 at physiological pH.

In certain aspects, in addition to the indicated substitutions, the RU may comprise additional substitutions as compared to SEQ ID NO:1. For example, the additional substitutions may be up to 1, up to 2, up to 3, up to 4, up to 5, up to 6, up to 7, up to 8, up to 9, or up to 10 conservative amino acid substitutions as compared to SEQ ID NO:1.

In certain aspects, the RU may comprise a 33-36 amino acid long sequence having a sequence at least 85%, at least 90%, at least 91%, at least 92%, at least 93%, at least 94%, at least 95%, or more identical to SEQ ID NO:1 and may comprise one or more of the substitutions that increase the overall positive charge of the repeat unit.

In certain aspects, the 33-36 long amino acid sequence of the repeat unit has at least 80% sequence identity (e.g., at least 85%, at least 90%, at least 91%, at least 92%, at least 93%, at least 94%, at least 95%, or more) to the amino acid sequence:

i.
(SEQ ID NO: 17)
LTPKQ VVAIA SX¹²X¹³GG KQALE TVQRL LPVLC QDHG
ii.
(SEQ ID NO: 18)
LTPRQ VVAIA SX¹²X¹³GG KQALE TVQRL LPVLC QDHG
iii.
(SEQ ID NO: 19)
LTPDQ VVAIA KX¹²X¹³GG KQALE TVQRL LPVLC QDHG
iv.
(SEQ ID NO: 20)
LTPDQ VVAIA RX¹²X¹³GG KQALE TVQRL LPVLC QDHG
v.
(SEQ ID NO: 21)
LTPDQ VVAIA SX¹²X¹³GG KQALE TVKRL LPVLC QDHG
vi.
(SEQ ID NO: 22)
LTPDQ VVAIA SX¹²X¹³GG KQALE TVRRL LPVLC QDHG
vii.
(SEQ ID NO: 23)
LTPDQ VVAIA SX¹²X¹³GG KQALE TVQRL LPVLK QDHG
viii.
(SEQ ID NO: 24)
LTPDQ VVAIA SX¹²X¹³GG KQALE TVQRL LPVLR QDHG
ix.
(SEQ ID NO: 25)
LTPDQ VVAIA SX₁₂X₁₃GG KQALE TVQRL LPVLC QKHG;
or
x.
(SEQ ID NO: 26)
LTPDQ VVAIA SX₁₂X₁₃GG KQALE TVQRL LPVLC QRHG,

wherein at least one of the amino acid residues at positions 4, 11, 23, and 32 has a positively charged side chain.

In certain aspects, the NBD may include a plurality of RUs ordered from N-terminus to C-terminus of the NBD to recognize a target nucleic acid. For example, the NBD may include 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 RUs, where at least one of the RUs is a RU as disclosed herein. In certain aspects, the NBD may include a plurality of RUs as disclosed herein. In certain aspects, the number of RUs as disclosed herein that may be included in a NBD may be determined by the net positive charge desired for the NBD and the net charge of each RU present in the NBD. In certain aspects, the desired net positive charge of the NBD may be at least +15, at least +20, at least +25, at least +30, at least +35, at least +40, at least +45, at least +50, at least +55, at least +60, or more. The number of the RUs as disclosed herein that may be included in the NBD may be at least 1, 2, 3, 4, 5, 6, 7, 8, 9, 10, 11, 12, 13, 14, or more. In certain aspects, the NBD may include one or more of the RUs disclosed herein and one or more RUs of naturally occurring transcription activator like effector (TALE) proteins, such as RUs from Xanthomonas or Ralstonia TALE proteins.

In certain aspects, the target nucleic acid may be DNA, i.e., the NBD may be a DNA-binding domain (DBD). In certain aspects, the amino acids present at positions 12 and 13 of the RUs may be selected based on the sequence of the target nucleic acid as is known for RUs from Xanthomonas or Ralstonia TALE proteins.

In certain aspects, the NBD may be associated with a functional domain. Such functional domains are further described herein. The NBD may be associated with a functional domain via a covalent interaction or via a non-covalent interaction. For example, a covalent interaction may involve conjugation of the NBD to a functional domain, e.g., a fusion protein comprising the NBD and the functional domain. A non-covalent interaction between a NBD as disclosed herein and a functional domain may involve use of binding members of a heterodimer as further explained in the next section. Briefly, the NBD may be conjugated to a first member of the heterodimer and the functional domain may be conjugated to second member of the heterodimer and the NBD and functional domain may interact via non-covalent interaction between the first and second members of the heterodimer. In certain aspects, the first member and or the second member may have a sequence that has a net positive charge (e.g., a net positive charge of at least +5, +10, +15, +20, +25, +30, or more which may then reduce the number of positively charged RUs required to impart a net positive charge on the NBD sufficient for making the NBD cell permeable.

In other aspects, instead of or in addition to the NBD including at least one non-naturally occurring RU having a net positive charge of at least +2, where the RU is derived from the sequence of SEQ ID NO:1 and includes at least one amino acid substitution as provided in the foregoing section, the NBD may include RUs derived from naturally occurring proteins comprising such RUs and selected because these RUs comprise an amino acid sequence that has a net charge of at least +2. In certain aspects, a recombinant polypeptide comprising a nucleic acid binding domain (NBD) and a heterologous functional domain is disclosed. The NBD comprising at least three repeat units (RUs) ordered from N-terminus to C-terminus of the NBD to specifically bind to a target nucleic acid, wherein each of the RUs comprises the sequence:

X_{1 to y}—X_{y+1}X_{y+2}—X_{(13 or 14)-(33 or 34 or 35)}, wherein

X_{1-y} is a chain of 10 or 11 contiguous amino acids, and y=10 or 11,

X_{y+1}X_{y+2} is a diresidue present at positions 11 and 12 or 12 and 13,

X_{(13 or 14) to (33 or 34 or 35)} is a chain of 21, 22 or 23 contiguous amino acids, starting at position 13, when the diresidue is present at positions 11 and 12 or starting at position 14, when the diresidue is present at positions 11 and 12,

the net charge of each of the RUs is at least +2, and

the net charge of the polypeptide is at least +30.

In certain aspects, the at least three RUs present in the NBD independently comprises a 33-36 amino acid long sequence that is at least 80% identical to one of:

(SEQ ID NO: 27)
LTPEQVVAIACNKGGKQALKTVQRLLPVLCKPPYC;
(SEQ ID NO: 28)
LTPNQVVAIASNKGGKQALETVQRLLPVLCKPPHR;
(SEQ ID NO: 29)
LTPKQVVAIAGYKGANQALGTVQRLLPVLCKPPYG;
(SEQ ID NO: 30)
LTPKQVVAIANYKGAKQALETVQRLLPLLCKPPYG;
(SEQ ID NO: 31)
LTPKQVVAIASYKGANQALGTVQRLLPVLCKPPYG;
(SEQ ID NO: 32)
MTPKQVVAIASYKGANQALGTVQRLLPVLCKPPYG;
(SEQ ID NO: 33)
LTNDRLVALACIGGRSALNAVKDGLPNALTLIRR;
(SEQ ID NO: 34)
LTPAQVVAIASHNGGKQALKTVQRLLPVLCQAHGL;
(SEQ ID NO: 35)
LVTGQLLKIAKRGGVNAVEAVHASRNALTGAPLH;
(SEQ ID NO: 36)
LTPDQVVAIASNGGGKQALETVRRLLPVLCKPPYR;
(SEQ ID NO: 37)
LTPDQVVAIASNGGGKQALKTVQRLLPVLCKPPYS;
(SEQ ID NO: 38)
LTPNQVVAIASNHGGKQALETVQRLLPVLRKPPYG;
(SEQ ID NO: 39)
LTPEQVVAIASNKGGKQALETVQRLLPVLRKPPYG;
(SEQ ID NO: 40)
LLPHQVVAIVSNSGGKQALETVRRLLPVLCKPPYS;
(SEQ ID NO: 41)
LTPKQVVAIASYGGKQALETVQRLLPVLCKPPYG;
(SEQ ID NO: 42)
LTPKQVVAIASYGGKQSLETVQRLLPVLCKPPYG;
(SEQ ID NO: 43)
LTPKQVVAIASYKGANQALETVQRLLPVLCKPPYG;
(SEQ ID NO: 44)
LTNDRLVALACIGGRSALNAVKDGLPNALTLITR;
(SEQ ID NO: 45)
LTPNQVVAIASGIGGRQALETVHRLLPVLCKPPYG;
(SEQ ID NO: 46)
LTPNQVVAIASHDGGKQALETVQRLLPVLRKPPYG;
(SEQ ID NO: 47)
LTPEQVVAIASHGGAKQALKTVQRLLPVLCQNHGL;
(SEQ ID NO: 48)
LTPEQVVAIASHNGGKQALETVQRLLPVLCKPPYR;
(SEQ ID NO: 49)
LTPKQVVAIASHNGGKQALETVQRLLPVLCHPPYG;
(SEQ ID NO: 50)
LTPKQVVAIASHNGGKQALETVQRLLPVLCQPPYG;
(SEQ ID NO: 51)
LTPNQVVAIASHNGGKQALETVQRLLPVLCKPPYG;
(SEQ ID NO: 52)
LTRNQVVAIASHNGGKQALETVQRLLPVLCKEYGL;
(SEQ ID NO: 53)
LTPEQVVAIASKGGGKQALETVQRLLPVLCKPAYG;
(SEQ ID NO: 54)
LTPNQVVAIASKGGGKQALETVQRLLPVLCQPPYG;
(SEQ ID NO: 55)
LTPDQVVAIASKIGGKQALETVQRLLPVLCKPPYG;
(SEQ ID NO: 56)
LTPAQVVAIASNGGGKQALETVRRLLPVLCQAHGL;
(SEQ ID NO: 57)
LTPARVVAIASNGGGKQALQTVQRLLPVLCEQHGL;
(SEQ ID NO: 58)
LTPDQVVAIASNGGAKQALKTVQRLLPVLCQPPYG;
(SEQ ID NO: 59)
LTPNQVIAIASNGGGKQALETVQRLLPVLCKPPYG;
(SEQ ID NO: 60)
LTPNQVVAIASNHGGKQALETVQRLLPVLCKPPYN;
(SEQ ID NO: 61)
LTPAKVVAIASNIGGKQALETVQRLLPVLCQAHGL;
(SEQ ID NO: 62)
LTPAQVVAIACNIGGKQALETVRRLLPVLCQAHGL;
(SEQ ID NO: 63)
LTPAQVVAIASNIGGKQALETVQRLLPVLCRAHGL;
(SEQ ID NO: 64)
LTPAQVVAIASNIGGKQALETVRRLLPVLCQAHGL;
(SEQ ID NO: 65)
LTPDQVVAIARNIGGKQALETVRRLLPVLCQAHGL;
(SEQ ID NO: 66)
LTPDQVVAIASNIGGKQALKTVQRLLPVLCQAHGL;
(SEQ ID NO: 67)
LTPEQVVTIANNIGGKQALETVQRLLPVLRKPPYG;
(SEQ ID NO: 68)
LTPNQVVTIANNIGGKQALETVQRLLPVLCKPPYG;
(SEQ ID NO: 69)
LTPEQVVAIASNKGGKQALETVQRLLPVLCKPPYG;
(SEQ ID NO: 70)
LTPAQVVAIASNNGGKQALERVQRLLPVLCQAHGL;
(SEQ ID NO: 71)
LTPAQVVAIASNNGGKQALETVRRLLPVLCQAHGL;
(SEQ ID NO: 72)
LTPNQVVAIASNNGAKQALETVQRLLPVLCKPPHP;
(SEQ ID NO: 73)
LTPNQVVAIASNNGGKQALETVQRLLPVLCKPAYG;
(SEQ ID NO: 74)
LTPNQVVAIASNNGGKQALETVQRLLPVLCKPPHP;
(SEQ ID NO: 75)
LTREQVVAIASNNGGKQALETVQRLLPVLRQAHGL;
(SEQ ID NO: 76)
LTRNQVVAIVNNNGGKQALETVHRLLPVLCQPPHG;
(SEQ ID NO: 77)
LTRNQVVAIVNNNGGKQALETVHRLLPVLCQPPYG;
(SEQ ID NO: 78)
LTPAQVVAIASNSGGKQALETVQRLLPVLRQAHGL;
(SEQ ID NO: 79)
LSPNQVVAIASHNGGKPALETVQRLLPVLCKPPY;
(SEQ ID NO: 80)
LLPDQVVAIVSNNGGKLALGTVQRLLPVLCKPPY;
(SEQ ID NO: 81)
LTPAQVVAIASNGGKQALETVRRLLPVLCQAHGL;
(SEQ ID NO: 82)
LTPAQVVAIASNSGGKPALETVRRLLPVLCQAHG;
(SEQ ID NO: 83)
LTPDQVIAIVSNGGGKPALETVRRLLPVLCKHPY;
(SEQ ID NO: 84)
LTPDQVIAIVSNGGGKPALETVRRLLPVLCKPPY;
(SEQ ID NO: 85)
LTPDQVVTIASNNGGKPALETVRRLLPVLCKPPY;
(SEQ ID NO: 86)
LTPNQVVAIASNNGGKPALETVQRLLPVLCKPPY;
(SEQ ID NO: 87)
LTPVQVVAIASNGGKQALATVQRLLPVLCQAHGL;
and
(SEQ ID NO: 88)
LTPKQVVAIASYGGKQALETVQRLLPVLCQPPYG.

In certain aspects, the at least three RUs present in the NBD each independently comprises a 33-36 amino acid long sequence that is at least 80% identical to one of:

(SEQ ID NO: 89)
LSTTRVVSIACIGGRQALKAIKTHMPALRQAPYS;
(SEQ ID NO: 90)
LSTTRVVSIACIGGRQALEAIKTHMPALRQAPYS;
(SEQ ID NO: 91)
LTPQQVVAIASNTGGKQALEAVTVQLRVLRGARYG;
(SEQ ID NO: 92)
LTPQQVVAIASNTGGKRALEAVCVQLPVLRAAPYR;
(SEQ ID NO: 93)
LSTAQVVAVAGRNGGKQALEAVRAQLPALRAAPYG;
(SEQ ID NO: 94)
LSIAQVVAVASRSGGKQALEAVRAQLLALRAAPYG;
(SEQ ID NO: 95)
LSTAQVVAVASGSGGKPALEAVRAQLLALRAAPY;
(SEQ ID NO: 96)
LSTAQVVAVASGSGGKQALEAVRVQLLALRAAPYG;
(SEQ ID NO: 97)
LSTAQVVAVASGSGGKPALEAVRAQLLALRAAPYG;
(SEQ ID NO: 98)
LSTAQVVAVASGSGGKPALEAVRAQLLALRAAPYG;
(SEQ ID NO: 99)
LNTAQVVAIASHDGGKPALEAVRAKLPVLRGVPYA;
(SEQ ID NO: 100)
LSTAQVVAVASHDGGKPALEAVRKQLPVLRGVPHQ;
(SEQ ID NO: 101)
LSTAQVVAVASHDGGKPALEAVRKQLPVLRGVPHQ;
(SEQ ID NO: 102)
LSTEQVVAIASHNGGKQALEAVKAQLPVLRRAPYG;
(SEQ ID NO: 103)
LSVAQVVTIASHNGGKQALEAVRAQLLALRAAPYG;
(SEQ ID NO: 104)
LNTAQVVAIASHYGGKPALEAVWAKLPVLRGVPYA;
(SEQ ID NO: 105)
LSTAQVVAIASNGGGKQALEGIGEQLRKLRTAPYG;
(SEQ ID NO: 106)
LSPEQVVAIASNHGGKQALEAVRALFRGLRAAPYG;
(SEQ ID NO: 107)
LSTEQVVAIASNHGGKQALEAVRALFRGLRAAPYG;
(SEQ ID NO: 108)
LSTEQVVAIASNKGGKQALEAVKAQLLALRAAPYA;
(SEQ ID NO: 109)
LSTEQVVAIASNNGGKQALEAVKAQLPVLRRAPCG;
(SEQ ID NO: 110)
LSTEQVVAIASNNGGKQALEAVKAQLPVLRRAPYG;
(SEQ ID NO: 111)
LSTEQVVAVASNNGGKQALKAVKAQLLALRAAPYE;
(SEQ ID NO: 112)
LSTAQLVAIASNPGGKQALEAIRALFRELRAAPYA;
(SEQ ID NO: 113)
LSTAQLVAIASNPGGKQALEAVRALFRELRAAPYA;
(SEQ ID NO: 114)
LSTAQLVAIASNPGGKQALEAVRAPFREVRAAPYA;
(SEQ ID NO: 115)
LSTAQLVSIASNPGGKQALEAVRALFRELRAAPYA;
(SEQ ID NO: 116)
LSTAQVVAIASNPGGKQALEAVRALFRELRAAPYA;
(SEQ ID NO: 117)
LTPQQVVAIASNTGGKRALEAVRVQLPVLRAAPYE;
(SEQ ID NO: 118)
LSTAQVVAIATRSGGKQALEAVRAQLLDLRAAPYG;
(SEQ ID NO: 119)
LSTAQVVAIASSHGGKQALEAVRALFRELRAAPYG;
(SEQ ID NO: 120)
LSTAQVATIASSIGGRQALEALKVQLPVLRAAPYG;
and
(SEQ ID NO: 121)
LSTAQVATIASSIGGRQALEAVKVQLPVLRAAPYG.

In certain aspects, the at least three RUs present in the NBD each independently comprises a 33-36 amino acid long sequence that is at least 80% identical to one of:

(SEQ ID NO: 122)
FRQADIVKIASNGGSAQALNAVIKLGPTLRQRG;
(SEQ ID NO: 123)
FRQADIVKMASNGGSAQALNAVIKLGPTLRQRG;
(SEQ ID NO: 124)
FRQTDIVKMAGSGGSAQALNAVIKHGPTLRQRG;
(SEQ ID NO: 125)
FNRADIVRIAGNGGGAQALYSVRDAGPTLGKRG;
(SEQ ID NO: 126)
FSRADIVRIAGNGGGAQALYSVLDVGPTLGKRG;
(SEQ ID NO: 127)
LQRADIVKIAGNGGGAQALQAVITHRAALTQAG;
(SEQ ID NO: 128)
FSATDIVKIASNIGGAQALQAVISRRAALIQAG;
(SEQ ID NO: 129)
FSAADIVKIASNNGGAQALQAVISRRAALIQAG;
and
(SEQ ID NO: 130)
FTLTDIVKMAGNNGGAQALKVVLEHGPTLRQRG.

In certain aspects, the at least three RUs present in the NBD each RU independently comprises a 33-36 amino acid long sequence that is at least 80% identical to one of:

(SEQ ID NO: 131)
FNTEQIVRMVSHDGGSLNLKAVKKYHDALRERK;
(SEQ ID NO: 132)
LDRQQILRIASHDGGSKNIAAVQKFLPKLMNFG;
(SEQ ID NO: 133)
FSAKHIVRIAAHIGGSLNIKAVQQAQQALKELG;
(SEQ ID NO: 134)
LGHKELIKIAARNGGGNNLIAVLSCYAKLKEMG;
(SEQ ID NO: 135)
FNAEQIVRMVSHKGGSKNLALVKEYFPVFSSFH;
(SEQ ID NO: 136)
FNAEQIVRMVSHKGGSKNLALVKEYFPVFSSFH;
and
(SEQ ID NO: 137)
FNAEQIVSMVSNGGGSLNLKAVKKYHDALKDRG.

In certain aspects, one of the at least three RUs present in the NBD may comprise a 33-36 amino acid long sequence that is at least 80% identical to:

LEPKDIVSIASHIGATQAITTLLNKWAALRAKG(SEQ ID NO: 138).

In certain aspects, one of the at least three RUs present in the NBD may comprise a 33-36 amino acid long sequence that is at least 80% identical to:

FNRASIVKIAGNSGGAQALQAVLKHGPTLDERG(SEQ ID NO: 139).

In certain aspects, RUs from two or more of the lists of naturally-occurring RUs may be combined in a single NBD.

In certain aspects, the NBD that has an overall positive charge of at least +15.

In certain aspects, the diresidues at positions 11 and 12 or at positions 12 and 13 of the foregoing RUs are independently selected from the following: HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, and S*, where (*) means that the amino acid is absent.

In certain aspects, one or more RUs in a NBD may be at least 80%, 85%, 90%, 91%, 92%, 93%, 94%, 95%, 96%, 97%, 98%, or 99%, or a 100% identical to a RU provided herein. Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

Percent Identity=(Matches×100)/Length of aligned region (with gaps)

The phrase “conservative amino acid substitution” refers to substitution of amino acid residues within the following groups: 1) L, I, M, V, F; 2) R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Guidance for substitutions, insertions, or deletions may be based on alignments of amino acid sequences of proteins from different species or from a consensus sequence based on a plurality of proteins having the same or similar function.

In certain aspects, the disclosed NBD may include a nuclear localization sequence (NLS) to facilitate entry into an organelle of a cell, e.g. the nucleus of a cell, e.g., an animal or a plant cell. In certain aspects, the disclosed NBD may include a half-RU or a partial RU that is 15-20 amino acid long sequence. Such a half-RU may be included after the last RU present in the NBD and may be derived from a RU identified in Xanthomonas or Ralstonia TALE protein. In certain aspects, the disclosed NBD may include an N-terminal domain. The N-terminal domain may be the N-cap domain or a fragment thereof from TALE proteins like those expressed in Burkholderia, Paraburkholderia, or Xanthomonas. In certain aspects, the disclosed NBD may include a C-terminal domain. The C-terminal domain may be a C-cap domain or a fragment thereof from TALE proteins like those expressed in Burkholderia, Paraburkholderia, or Xanthomonas.

### Positively Charged Heterodimer Pairs

The present disclosure provides binding members of a heterodimer pair that have been modified by amino acid substitution to introduce positively charged amino acids thereby increasing the positive charge of the binding members.

In certain aspects, the binding members of a heterodimer pair are referred to as 37A and 37B. The sequences of the unmodified proteins 37A and 37B are as follows:

37A_Unmodified:
(SEQ ID NO: 2)
DSDEHLKKLKTFLENLRRHLDRLDKHIKQLRDILSENPEDERVKDVIDLSE
RSVRIVKTVIKIFEDSVRKKE
37B_Unmodified:
(SEQ ID NO: 3)
MDDKELDKLLDTLEKILQTATKIIDDANKLLEKLRRSERKDPKVVETYVE
LLKRHEKAVKELLEIAKTHAKKVE

The underlined residues indicate amino acids that can be substituted with an amino acid with a positively charged side chain, e.g., K, R, or H, without significantly reducing dimerization of 37A and 37B.

In certain aspects, 1-14, e.g., 3-14, 5-14, 8-14, 5-12, 5-9, such as, 3, 5, 8, 9, 12, or 14 amino acids of the 37A protein may be substituted with an amino acid with a positively charged side chain. For example, a positively charged first member of a heterodimer pair may have an amino acid sequence that is about 72 amino acids long and is at least 75% identical to the sequence of the unmodified 37A protein (SEQ ID NO:2) and comprises at least one of the following amino acid substitutions relative to the sequence of the unmodified 37A protein: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H.

In certain aspects, a positively charged first member of a heterodimer pair may have an amino acid sequence that is at least 75% identical (e.g., at least 80%) to the sequence of the unmodified 37A protein (SEQ ID NO:2) and comprises at least one, at least two, at least three, at least four, at least five, at least six, at least seven, at least eight, at least nine, at least ten, at least eleven, at least twelve, at least thirteen, or all of the following amino acid substitutions relative to the sequence of the unmodified 37A protein: D3K; E4K; T11K; D24K; D32K; S35K; E39K; D40K; E41K; D45K; D48K; L49K; T59K; and D66K. In certain aspects, a positively charged first member of a heterodimer pair may have the amino acid sequence of SEQ ID NO:2 but with at least one, at least two, at least three, at least four, at least five, at least six, at least seven, at least eight, at least nine, at least ten, at least eleven, at least twelve, at least thirteen, or all of the following amino acid substitutions relative to the sequence of SEQ ID NO:2: D3K; E4K; T11K; D24K; D32K; S35K; E39K; D40K; E41K; D45K; D48K; L49K; T59K; and D66K.

In certain aspects, a positively charged 37A protein may have an amino acid sequence as follows:

(SEQ ID NO: 4)
DSDEHLKKLKKFLENLRRHLDRLKKHIKQLRDILSENPEDKRVKDVIDLS
ERSVRIVKTVIKIFEDSVRKKE;
(SEQ ID NO: 5)
DSKEHLKKLKKFLENLRRHLDRLKKHIKQLRKILSENPEDKRVKDVIDLS
ERSVRIVKTVIKIFEDSVRKKE;
(SEQ ID NO: 6)
DSKKHLKKLKKFLENLRRHLDRLKKHIKQLRKILKENPEDKRVKDVIDLS
ERSVRIVKKVIKIFEDSVRKKE;
(SEQ ID NO: 7)
DSKKHLKKLKKFLENLRRHLDRLKKHIKQLRKILKENPEDKRVKDVIDKS
ERSVRIVKKVIKIFEDSVRKKE;
(SEQ ID NO: 8)
DSKKHLKKLKKFLENLRRHLDRLKKHIKQLRKILKENPKDKRVKDVIDK
SERSVRIVKKVIKIFEKSVRKKE;
or
(SEQ ID NO: 9)
DSKKHLKKLKKFLENLRRHLDRLKKHIKQLRKILKENPKKKRVKKVIKK
SERSVRIVKKVIKIFEKSVRKKE;.

Amino acid substitutions relative to the unmodified 37A protein are indicated by underlining.

In certain aspects, 1-13, e.g., 3-9, 5-9, or 8-9, such as, 3, 5, 7, 8, or 9 amino acids of the 37B protein may be substituted with an amino acid with a positively charged side chain e.g., K, R, or H. For example, a positively charged first member of a heterodimer pair may have an amino acid sequence that is about 74 amino acids long and is at least 75% identical (e.g., at least 80% or 85% identical) to the sequence of the unmodified 37B protein (SEQ ID NO:3) and comprises at least one (e.g., at least 2, at least 3, at least 4, at least 5, at least 6, at least 7, at least 8, at least 9, at least 10, at least 11, at least 12, or all) of the following amino acid substitutions relative to the sequence of the unmodified 37B protein: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.

In certain aspects, a positively charged second member of a heterodimer pair may have the amino acid sequence of SEQ ID NO:3 but with at least one (e.g., at least 2, at least 3, at least 4, at least 5, at least 6, at least 7, at least 8, at least 9, at least 10, at least 11, at least 12, or all) of the following amino acid substitutions relative to the sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H.

In certain aspects, a positively charged 37B protein may have an amino acid sequence as follows:

(SEQ ID NO: 10)
MKDKELDKLLDTLEKILQKATKIIDDANKLLEKLRRSERKKPKVVETYVE
LLKRHEKAVKELLEIAKTHAKKVE;
(SEQ ID NO: 16)
MDDKKLDKLLDKLEKILQTATKIIDDANKLLEKLRRSERKDPKVVKTYV
ELLKRHEKAVKELLEIAKTHAKKVE;
(SEQ ID NO: 12)
MKDDKELDKLLDTLEKILQTATKIIDKANKLLEKLRRSKRKDPKVVETY
VELLKRHEKAVKELLEIAKKHAKKVE;
(SEQ ID NO: 13)
MKDKELDKLLDKLEKILQKATKIIDKANKLLEKLRRSERKKPKVVKTYV
ELLKRHEKAVKELLEIAKTHAKKVE;
(SEQ ID NO: 14)
MKDKKLDKLLDKLEKILQKATKIIDKANKLLEKLRRSKRKKPKVVKTYV
ELLKRHEKAVKELLEIAKTHAKKVE;
or
(SEQ ID NO: 15)
MKKDKKLDKLLDKLEKILQKATKIIDKANKLLEKLRRSKRKKPKVVKTY
VELLKRHEKAVKELLEIAKTHAKKVE.

Amino acid substitutions relative to the unmodified 37B protein are indicated by underlining.

In certain aspects, a positively charged first binding member or positively charged second binding member of a heterodimer may be fused to a nucleic acid binding domain or a functional domain. For example, a positively charged first binding member may be fused to a nucleic acid binding domain and a positively charged second binding member of the heterodimer may be fused to a functional domain. The nucleic acid binding domain (NBD) and the functional domain may be as described herein or as are known in the art. The first or the second member may be fused to the N- or the C-terminus of the NBD or the functional domain. In certain aspects, the NBD may be a transcription activator-like effector (TALE), modular animal pathogen nucleic acid binding domain, zinc finger protein, or single-guide RNA. Modular animal pathogen nucleic acid binding domain may be derived from DNA binding RUs identified in proteins from animal pathogens, such as, Legionella quateirensis, Burkholderia, Paraburkholderia, or Francisella.

In certain aspects, instead of or in addition to substituting in amino acids with positively charged side chain in the sequence of a first binding member and/or a second binding member of a heterodimer as disclosed herein, a binding member of a heterodimer may be fused to a nucleic acid binding domain or a functional domain via a linker. In certain aspects, the linker may be GSGGGGG. In certain aspects, the linker may be a positively charged linker that includes at least 4, at least 5, or at least 6 amino acids with a positively charged side chain. In certain aspects, a positively charged linker may have the sequence: GKGSKGKGKGK (SEQ ID NO: 140) or GKGSKGKGKGKGSK (SEQ ID NO: 141).

In certain aspects, a first or a second binding member of a heterodimer may be conjugated to the N- or C-terminus of a nucleic acid binding domain or a functional domain with or without a linker. The linker, if present, may have a net neutral charge or may have a net positive charge.

In certain aspects, a heterodimer comprising the first binding member and the second binding member as provided herein is disclosed. The first binding member and/or the second binding member may be fused to a NBD or a functional domain.

In certain aspects, the heterodimer may include a first binding member and a second binding member as provided herein, where the first binding member is fused to a functional domain (e.g., to the N-terminus of the functional domain) and the second binding member is fused to a DNA binding domain (e.g., to the C-terminus of the DNA binding domain).

In certain aspects, the heterodimer may include a first binding member and a second binding member as provided herein, where the second binding member is fused to a functional domain (e.g., to the N-terminus of the functional domain) and the first binding member is fused to a DNA binding domain e.g., to the C-terminus of the DNA binding domain).

In certain aspects, the first binding member as disclosed herein comprises a net charge of at least +15 (e.g., at least +20, +25, +30, or more). In certain aspects, the second binding member comprises a net charge of at least +15 (e.g., at least +20, +25, +30, or more). In certain aspects, the first binding member and the second binding member each comprise a net charge of at least +15 (e.g., at least +20, +25, +30, or more).

Also provide herein are sequences of a positively charged KRAB domain that is cell permeable. In certain aspects, a positively charged KRAB domain may have an amino acid sequence at least 80%, at least 90%, or at least 95% identical to the amino acid sequence of:

>37B-linker-KRAB-net5-1
(SEQ ID NO: 142)
MKDKELDKLLDTLEKILQKATKIIDDANKLLEKLRRSERKKPKVVETYVEL
LKRHEKAVKELLEIAKTHAKKVEGSGGGGGMDAKSLTAWSRTLVTFKDVFV
DFTREEWKLLDTAQQIVYRNVMLENYKNLVSLGYQLTKPDVILRLEKGEEP
>37B-linker-KRAB-net5-2
(SEQ ID NO: 143)
MDDKKLDKLLDKLEKILQTATKIIDDANKLLEKLRRSERKDPKVVKTYVEL
LKRHEKAVKELLEIAKTHAKKVEGSGGGGGMDAKSLTAWSRTLVTFKDVFV
DFTREEWKLLDTAQQIVYRNVMLENYKNLVSLGYQLTKPDVILRLEKGEEP
>37B-linker-KRAB-net5-3
(SEQ ID NO: 144)
MKDDKELDKLLDTLEKILQTATKIIDKANKLLEKLRRSKRKDPKVVETYVE
LLKRHEKAVKELLEIAKKHAKKVEGSGGGGGMDAKSLTAWSRTLVTFKDVF
VDFTREEWKLLDTAQQIVYRNVMLENYKNLVSLGYQLTKPDVILRLEKGEE
P
>37B-linker-KRAB-net10
(SEQ ID NO: 145)
MKDKELDKLLDKLEKILQKATKIIDKANKLLEKLRRSERKKPKVVKTYVEL
KRHEKAVKELLEIAKTHAKKVEGSGGGGGMDAKSLTAWSRTLVTFKDVFVD
FTREEWKLLDTAQQIVYRNVMLENYKNLVSLGYQLTKPDVILRLEKGEEP
>37B-linker-KRAB-net15
(SEQ ID NO: 146)
MKDKKLDKLLDKLEKILQKATKIIDKANKLLEKLRRSKRKKPKVVKTYVEL
LKRHEKAVKELLEIAKTHAKKVEGSGGGGGMDAKSLTAWSRTLVTFKDVFV
DFTREEWKLLDTAQQIVYRNVMLENYKNLVSLGYQLTKPDVILRLEKGEEP
>37B-linker-KRAB-net20
(SEQ ID NO: 147)
MKKDKKLDKLLDKLEKILQKATKIIDKANKLLEKLRRSKRKKPKVVKTYVE
LLKRHEKAVKELLEIAKTHAKKVEGKGSKGKGKGKMDAKSLTAWSRTLVTF
KDVFVDFTREEWKLLDTAQQIVYRNVMLENYKNLVSLGYQLTKPDVILRLE
KGEEP

The amino acid substitutions relative to the unmodified 37B protein are underlined; linker sequence is in bold font; and KRAB sequence is italicized.

In certain aspects, instead of using the 37A and 37B proteins (or modified variants thereof) to mediate interaction between a nucleic acid binding domain and a functional domain, the binding members A1::B1; A2::B2; A3::B3; A4::B4, and A5::B5 of a heterodimer may be used. Sequences for these heterodimers are as follows:

A1:
(SEQ ID NO: 148)
PTDEVIEVLKELLRIHRENLRVNEEIVEVNERASRVTDREELERLLRRSNE
LIKRSRELNEESKKLIEKLERLAT;
and
B1:
(SEQ ID NO: 149)
DNEEIIKEARRVVEEYKKAVDRLEELVRRAENAKHASEKELKDIVREILRI
SKELNKVSERLIELWERSQERAR;
or
A2:
(SEQ ID NO: 150)
TAEELLEVHKKSDRVTKEHLRVSEEILKVVEVLTRGEVSSEVLKRVLRKLE
ELTDKLRRVTEEQRRVVEKLN;
and
B2:
(SEQ ID NO: 151)
DLEDLLRRLRRLVDEQRRLVEELERVSRRLEKAVRDNEDERELARLSREHS
DIQDKHDKLAREILEVLKRLLERTE;
or
A3:
(SEQ ID NO: 152)
PEDDVVRIIKEDLESNREVLREQKEIHRILELVTRGEVSEEAIDRVLKRQE
DLLKKQKESTDKARKVVEERR;
and
B3:
(SEQ ID NO: 153)
DEVRLITEWLKLSEESTRLLKELVELTRLLRNNVPNVEEILREHERISREL
ERLSRRLKDLADKLERTRR;
or
A4:
(SEQ ID NO: 154)
DEEDHLKKLKTHLEKLERHLKLLEDHAKKLEDILKERPEDSAVKESIDELR
RSIELVRESIEIFRQSVEEEE;
and
B4:
(SEQ ID NO: 155)
GDVKELTKILDTLTKILETATKVIKDATKLLEEHRKSDKPDPRLIETHKKL
VEEHETLVRQHKELAEEHLKRTR;
or
A5:
(SEQ ID NO: 156)
MKKDKKLDKLLDKLEKILQKATKIIDKANKLLEKLRRSKRKKPKVVKTYVE
LLKRHEKAVKELLEIAKTHAKKVE;
and
B5:
(SEQ ID NO: 157)
MKKDKKLDKLLDKLEKILQKATKIIDKANKLLEKLRRSKRKKPKVVKTYVE
LLKRHEKAVKELLEIAKTHAKKVE.

In certain aspects, one or both binding members may include amino acid substitutions replacing an amino acid with a neutral or a negatively charged side chain with K, R, or H. In certain aspects, a first binding member may be conjugated to a nucleic acid binding domain and a second binding member of the same binding pair may be conjugated to a functional domain via a positively charged linker.

### Functional Domains

A NBD as disclosed herein can be associated with a functional domain as described in the preceding sections. The functional domain can provide different types of activity, such as genome editing, gene regulation (e.g., activation or repression), or visualization of a genomic locus via imaging. In certain aspects, the functional domain is heterologous to the NBD. Heterologous in the context of a functional domain and a NBD as used herein indicates that these domains are derived from different sources and do not exist together in nature.

A. Genome Editing Domains

A NBD as disclosed herein can be associated with a nuclease, wherein the NBD provides specificity and targeting and the nuclease provides genome editing functionality. In some embodiments, the nuclease can be a cleavage half domain, which dimerizes to form an active full domain capable of cleaving DNA. In other embodiments, the nuclease can be a cleavage domain, which is capable of cleaving DNA without needing to dimerize. For example, a nuclease comprising a cleavage half domain can be an endonuclease, such as FokI or Bfil. In some embodiments, two cleavage half domains (e.g., FokI or Bfil) can be fused together to form a fully functional single cleavage domain. When half cleavage domains are used as the nuclease, two MAP-NBDs can be engineered, the first MAP-NBD binding to a top strand of a target nucleic acid sequence and comprising a first FokI cleavage half domain and a second MAP-NBD binding to a bottom strand of a target nucleic acid sequence and comprising a second FokI half cleavage domain. In some embodiments, the nuclease can be a type IIS restriction enzyme, such as FokI or Bfil.

In some embodiments, a cleavage domain capable of cleaving DNA without need to dimerize may be a meganuclease. Meganucleases are also referred to as homing endonucleases. In some embodiments, the meganuclease may be I-Anil or I-OnuI.

A nuclease domain fused to a NBD can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to a NBD as disclosed herein can be a restriction endonuclease (or restriction enzyme). In some instances, a restriction enzyme cleaves DNA at a site removed from the recognition site and has a separate binding and cleavage domains. In some instances, such a restriction enzyme is a Type IIS restriction enzyme.

A nuclease domain fused to a NBD as disclosed herein can be a Type IIS nuclease. A Type IIS nuclease can be FokI or Bfil. In some cases, a nuclease domain fused to a MAP-NBD (e.g., L. quateirensis, Burkholderia, Paraburkholderia, or Francisella-derived) is FokI. In other cases, a nuclease domain fused to a MAP-NBD (e.g., L. quateirensis, Burkholderia, Paraburkholderia, or Francisella-derived) is Bfil.

FokI can be a wild-type FokI or can comprise one or more mutations. In some cases, FokI can comprise 1, 2, 3, 4, 5, 6, 7, 8, 9, 10, or more mutations. A mutation can enhance cleavage efficiency. A mutation can abolish cleavage activity. In some cases, a mutation can modulate homodimerization. For example, FokI can have a mutation at one or more amino acid residue positions 446, 447, 479, 483, 484, 486, 487, 490, 491, 496, 498, 499, 500, 531, 534, 537, and 538 to modulate homodimerization.

In some instances, a FokI cleavage domain is, for example, as described in Kim et al. “Hybrid restriction enzymes: Zinc finger fusions to Fok I cleavage domain,” PNAS 93: 1156-1160 (1996). In some cases, a FokI cleavage domain described herein is a FokI of SEQ ID NO: 11 (TABLE 2). In other instances, a FokI cleavage domain described herein is a FokI, for example, as described in U.S. Pat. No. 8,586,526.

TABLE 2 illustrates an exemplary FokI sequence that can be used herein with a method or system described herein.

| SEQ ID NO | FokI Sequence |
|---|---|
| SEQ ID NO: 11 | QLVKSELEEKKSELRHKLKYVPHEYIELIEIARNST |
| | QDRILEMKVMEFFMKVYGYRGKHLGGSRKPDGAIYT |
| | VGSPIDYGVIVDTKAYSGGYNLPIGQADEMQRYVEE |
| | NQTRNKHINPNEWWKVYPSSVTEFKFLFVSGHFKGN |
| | YKAQLTRLNHITNCNGAVLSVEELLIGGEMIKAGTL |
| | TLEEVRRKFNNGEINF |

A NBD can be linked to a functional group that modifies DNA nucleotides, for example an adenosine deaminase.

B. Regulatory Domains

As another example, NBD as disclosed herein can be linked to a gene regulating domain. A gene regulation domain can be an activator or a repressor. For example, a NBD as disclosed herein can be linked to an activation domain, such as VP16, VP64, p65, p300 catalytic domain, TET1 catalytic domain, TDG, Ldb1 self-associated domain, SAM activator (VP64, p65, HSF1), or VPR (VP64, p65, Rta). The terms “activator,” “activation domain” and “transcriptional activator” are used interchangeably to refer to a polypeptide that increases expression of a gene. Alternatively, a NBD can be linked to a repressor, such as KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. The terms “repressor,” “repressor domain,” and “transcriptional repressor” are used herein interchangeably to refer to a polypeptide that decreases expression of a gene.

In some embodiments, a NBD as disclosed herein can be linked to a DNA modifying protein, such as DNMT3a. A NBD can be linked to a chromatin-modifying protein, such as lysine-specific histone demethylase 1 (LSD1). A NBD can be linked to a protein that is capable of recruiting other proteins, such as KRAB. The DNA modifying protein (e.g., DNMT3a) and proteins capable of recruiting other proteins (e.g., KRAB) can serve as repressors of transcription. Thus, NBD linked to a DNA modifying protein (e.g., DNMT3a) or a domain capable of recruiting other proteins (e.g., KRAB, a domain found in transcriptional repressors, such as Kox1) can provide gene repression functionality, can serve as transcription factors, wherein the NBD provides specificity and targeting and the DNA modifying protein and the protein capable of recruiting other proteins provides gene repression functionality, which can be referred to as an engineered genomic regulatory complex or a NBD-gene regulator (NBD-GR) and, more specifically, as a NBD-transcription factor (NBD-TF).

In some embodiments, expression of the target gene can be reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 95%, at least 97%, or at least 99% by using a DNA binding domain fused to a repression domain (e.g., a MAP-NBD-TF) of the present disclosure as compared to non-treated cells. In some embodiments, expression of a checkpoint gene can be reduced by over 90% by using a MAP-NBD-TF of the present disclosure as compared to non-treated cells.

In some embodiments, repression of the target gene with a DNA binding domain fused to a repression domain (e.g., a NBD-TF) of the present disclosure and subsequent reduced expression of the target gene can last for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days. In some embodiments, repression of the target gene with a MAP-NBD-TF of the present disclosure and subsequent reduced expression of the target gene can last for 1 days to 3 days, 3 days to 5 days, 5 days to 7 days, 7 days to 9 days, 9 days to 11 days, 11 days to 13 days, 13 days to 15 days, 15 days to 17 days, 17 days to 19 days, 19 days to 21 days, 21 days to 23 days, 23 days to 25 days, or 25 days to 28 days.

In various aspects, the present disclosure provides a method of identifying a target binding site in a target gene of a cell, the method comprising: (a) contacting a cell with an engineered transcriptional repressor comprising a DNA binding domain, a repressor domain, and a linker; (b) measuring expression of the target gene; and (c) determining expression of the target gene is repressed by at least 50%, at least 60%, at least 70%, at least 80%, at least 85%, at least 90%, at least 92%, at least 95%, at least 97%, or at least 99% for at least 3 days, wherein the target gene is selected from: a checkpoint gene and a T cell surface receptor.

In some aspects, expression of the target gene is repressed in at least 75%, at least 80%, at least 85%, at least 90%, at least 95%, or at least 99% of a plurality of the cells. In some aspects, the engineered genomic regulatory complex is undetectable after at least 3 days. In some aspects, determining the engineered genomic regulatory complex is undetectable is measured by qPCR, imaging of a FLAG-tag, or a combination thereof. In some aspects, the measuring expression of the target gene comprises flow cytometry quantification of expression of the target gene.

In some embodiments, repression of the target gene with a DNA binding domain fused to a repression domain (e.g., a NBD-TF) of the present disclosure can last even after the DNA binding domain-TF becomes undetectable. The DNA binding domain fused to a repression domain (e.g., a NBD-TF) can become undetectable after at least 3 days. In some embodiments, the DNA binding domain fused to a repression domain (e.g., a NBD-TF) can become undetectable after at least 1 day, at least 2 days, at least 3 days, at least 4 days, at least 5 days, at least 6 days, at least 1 week, at least 2 weeks, at least 3 weeks, or at least 4 weeks. In some embodiments, qPCR or imaging via the FLAG-tag can be used to confirm that the DNA binding domain fused to a repression domain (e.g., a NBD-TF) is no longer detectable.

C. Imaging Moieties

In certain aspects, the functional domain may be an imaging domain, e.g., a fluorescent protein, biotinylation reagent, tag (e.g., 6×-His or HA). A NBD can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY.

### Targets

In some aspects, described herein include methods of modifying the genetic material of a target cell utilizing a NBD described herein. A target cell can be a eukaryotic cell or a prokaryotic cell. A target cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtle, lizard or snake. A fish cell can be from a tropical fish. For example, the fish cell can be from a zebrafish (e.g., Danio rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include an epithelial cell, connective tissue cell, hormone secreting cell, a nerve cell, a skeletal muscle cell, a blood cell, an immune system cell, or a stem cell.

Exemplary mammalian cells can include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, PC12 cell line, primary cells (e.g., from a human) including primary T cells, primary hematopoietic stem cells, primary human embryonic stem cells (hESCs), and primary induced pluripotent stem cells (iPSCs).

In some embodiments, a NBD of the present disclosure can be used to modify a target cell. The target cell can itself be unmodified or modified. For example, an unmodified cell can be edited with a NBD of the present disclosure to introduce an insertion, deletion, or mutation in its genome. In some embodiments, a modified cell already having a mutation can be repaired with a NBD of the present disclosure.

In some instances, a target cell is a cell comprising one or more single nucleotide polymorphism (SNP). In some instances, a NBD-nuclease described herein is designed to target and edit a target cell comprising a SNP.

In some cases, a target cell is a cell that does not contain a modification. For example, a target cell can comprise a genome without genetic defect (e.g., without genetic mutation) and a NBD-nuclease described herein can be used to introduce a modification (e.g., a mutation) within the genome.

In some cases, a target cell is a cancerous cell. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenström's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, described herein include methods of modifying a target gene utilizing a NBD described herein. In some embodiments, genome editing can be performed by fusing a nuclease of the present disclosure with a DNA binding domain for a particular genomic locus of interest. Genetic modification can involve introducing a functional gene for therapeutic purposes, knocking out a gene for therapeutic gene, or engineering a cell ex vivo (e.g., HSCs or CAR T cells) to be administered back into a subject in need thereof. For example, the genome editing complex can have a target site within PDCD1, CTLA4, LAG3, TET2, BTLA, HAVCR2, CCR5, CXCR4, TRA, TRB, B2M, albumin, HBB, HBA1, TTR, NR3C1, CD52, erythroid specific enhancer of the BCL11A gene, CBLB, TGFBR1, SERPINA1, HBV genomic DNA in infected cells, CEP290, DMD, CFTR, IL2RG, CS-1, or any combination thereof. In some embodiments, a genome editing complex can cleave double stranded DNA at a target site in order to insert a chimeric antigen receptor (CAR), alpha-L iduronidase (IDUA), iduronate-2-sulfatase (IDS), or Factor 9 (F9). Cells, such as hematopoietic stem cells (HSCs) and T cells, can be engineered ex vivo with the genome editing complex. Alternatively, genome editing complexes can be directly administered to a subject in need thereof.

### Compositions

In certain aspects, the polypeptides described herein may be present in a pharmaceutical composition comprising a pharmaceutically acceptable excipient. In certain aspects, the polypeptides are present in a therapeutically effective amount in the pharmaceutical composition. A therapeutically effective amount can be determined based on an observed effectiveness of the composition. A therapeutically effective amount can be determined using assays that measure the desired effect in a cell, e.g., in a reporter cell line in which expression of a reporter is modulated in response to the polypeptides of the present disclosure. The pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Suitable pharmaceutically acceptable or physiologically acceptable diluents, carriers or excipients include, but are not limited to, nuclease inhibitors, protease inhibitors, a suitable vehicle such as physiological saline solution or citrate buffered saline.

### Delivery

The positively charged polypeptides disclosed herein and compositions comprising the disclosed polypeptides can be delivered into a target cell by any suitable means, including, for example, by contacting the cell with the polypeptide. In certain aspects, the positively charged polypeptides can be delivered into cells in a particular tissue (e.g., a solid tumor) by injecting a composition comprising the positively charged polypeptide directly into the solid tumor.

In other aspects, administration involves systemic administration (e.g., intravenous, intraperitoneal, intramuscular, subdermal, or intracranial infusion), direct injection (e.g., intrathecal), or topical application, etc.

### Methods

The present invention also provides a method of introducing a polypeptide having a net positive charge of at least +15 (e.g., at least +20, at least +25, at least +30, at least +35, at least +40, at least +45, at least +50, at least +55, at least +60, or more) with or without an agent associated with the positively charged polypeptide into a cell. The method comprises contacting the positively charged polypeptide, or a positively charged polypeptide and an agent associated with the positively charged polypeptide (e.g., where the agent is negatively charged and associates with the positively charged polypeptide via electrostatic interaction) with the cell, e.g., under conditions sufficient to allow penetration of the positively charged polypeptide, or an agent associated with the positively charged polypeptide, into the cell, thereby introducing a the positively charged polypeptide, or an agent associated with the positively charged polypeptide, or both, into a cell. In certain aspects, introduction of the positively charged polypeptide may be assessed by assaying the cell for presence of a signal indicative of the entry or assaying for an effect of the positively charged polypeptide in the cell.

In certain embodiments, the contact is performed in vitro. In certain embodiments, the contact is performed in vivo, e.g., in the body of a subject, e.g., a human or other animal or ex vivo. In one in vivo embodiment, sufficient positively charged polypeptide is present in the cell to provide a detectable effect in the subject, e.g., a therapeutic effect. In one in vivo embodiment, sufficient positively charged polypeptide is present in the cell to allow imaging of one or more penetrated cells or tissues. In certain embodiments, the observed or detectable effect arises from cell penetration.

The desired modifications or mutations in a polypeptide may be accomplished using any techniques known in the art. Recombinant DNA techniques for introducing such changes in a protein sequence are well known in the art. In certain embodiments, the modifications are made by site-directed mutagenesis of the polynucleotide encoding the protein. Other techniques for introducing mutations are discussed in Molecular Cloning: A Laboratory Manual, 2nd Ed., ed. by Sambrook, Fritsch, and Maniatis (Cold Spring Harbor Laboratory Press: 1989); the treatise, Methods in Enzymology (Academic Press, Inc., N.Y.); Ausubel et al. Current Protocols in Molecular Biology (John Wiley & Sons, Inc., New York, 1999). The modified protein is expressed and tested. In certain embodiments, a series of variants is prepared, and each variant is tested to determine its biological activity and its stability. The variant chosen for subsequent use may be the most stable one, the most active one, or the one with the greatest overall combination of activity and stability. After a first set of variants is prepared an additional set of variants may be prepared based on what is learned from the first set. Variants are typically created and overexpressed using recombinant techniques known in the art.

The polypeptide provided herein may be modified to increase yield, half-life, activity of the polypeptide. Such modifications include, PEGylation, glycosylation, lipidation, conjugation to Fc portion of human IgG, maltose binding proteins, albumin and the like. In certain aspects, the polypeptides (e.g., the NBDs, functional domains, conjugates thereof, and the like) provided herein may be fused to a peptide that enhances endosome degradation or lysis of the endosome to reduce sequestration of the polypeptides in the endosomes. In certain embodiments, the peptide is hemagglutinin 2 (HA2) peptide which is known to enhance endosome degradation.

A method of modulating expression of an endogenous gene in a cell is also provided. The method may include contacting the cell with the positively charged polypeptide as provided herein, wherein the polypeptide penetrates the cell membrane and wherein the NBD of the polypeptide binds to a target nucleic acid sequence present in the endogenous gene and the heterologous functional domain modulates expression of the endogenous gene. The nucleic acid may be a ribonucleic acid (RNA) or a deoxyribonucleic acid (DNA).

The functional domain may be a transcriptional activator and the target nucleic acid sequence is present in an expression control region of the gene, wherein the polypeptide increases expression of the gene. The transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

In other aspects, the functional domain is a transcriptional repressor and the target nucleic acid sequence is present in an expression control region of the gene, wherein the polypeptide decreases expression of the gene. The transcriptional repressor may be KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

The an endogenous gene may be a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAl gene, a TTR gene, a NR3Cl gene, a CD52 gene, an erythroid specific enhancer of the ECLllA gene, a CELE gene, a TGFERl gene, a SERPINAl gene, a HEV genomic DNA in infected cells, a CEP290 gene, a DMD gene, a CFTR gene, or an IL2RG gene.

The expression control region of the gene may include a promoter region of the gene.

The functional domain may be a nuclease comprising a cleavage domain or a half-cleavage domain and the endogenous gene is inactivated by cleavage.

In certain aspects, the polypeptide is a first polypeptide that binds to a first target nucleic acid sequence in the gene and comprises a half-cleavage domain and the method comprises introducing a second polypeptide that binds to a second target nucleic acid sequence in the gene and comprises a half-cleavage domain. The first target nucleic acid sequence and the second target sequence may be spaced apart in the gene and the two half-cleavage domains mediate a cleavage of the gene sequence at a location in between the first and second target nucleic acid sequences. The cleavage domain or the cleavage half domain may be FokI or Bfil, or a meganuclease.

The target gene may be any gene of interest, such as, those disclosed herein.

In certain aspects, a method of introducing an exogenous nucleic acid into a region of interest in the genome of a cell is provided. The method may include introducing into the cell a positively charged polypeptide comprising a NBD as disclosed herein, where the NBD of the polypeptide binds to the target nucleic acid sequence present adjacent the region of interest; and the exogenous nucleic acid, wherein the cleavage domain or the half-cleavage domain introduces a cleavage in the region of interest and wherein the exogenous nucleic acid in integrated into the cleaved region of interest by homologous recombination.

In certain aspects, introducing the polypeptide into the cell comprises contacting the cell with the polypeptide in absence of a transfection agent, wherein the polypeptide penetrates the cell membrane. In certain aspects, introducing the polypeptide and the exogenous nucleic acid into the cell comprises contacting the cell with a composition comprising the polypeptide associated with the exogenous nucleic acid, wherein the polypeptide penetrates the cell membrane and transports the exogenous nucleic acid into the cell. The cell may be any cell of interest, such as, those disclosed herein and the introducing may be performed in vivo, ex vivo or in vitro. In certain aspects, the introducing comprises administering the polypeptide to a subject. The administering may comprise parenteral administration. The administering may comprise intravenous, intramuscular, intrathecal, or subcutaneous administration. The administering may comprise direct injection into a site in a subject. The administering may comprise direct injection into a tumor, e.g., a solid tumor.

A method of modulating expression of an endogenous gene in a cell is disclosed, the method may include introducing into the cell the first binding member and the second binding member or a heterodimer as provided herein, wherein at least one of the first and second binding members penetrates the cell membrane and wherein the NBD binds to a target nucleic acid sequence present in the endogenous gene and the heterologous functional domain modulates expression of the endogenous gene.

In certain aspects, introducing into the cell the first and second binding members comprises contacting the cell with the first and second binding members. In certain aspects, introducing into the cell the first and second binding members comprises contacting the cell with the first binding member and introducing into the cell a nucleic acid encoding the second binding member. In certain aspects, introducing into the cell the first and second binding members comprises contacting the cell with the second binding member and introducing into the cell a nucleic acid encoding the first binding member. The nucleic acid encoding the first or second binding member may be RNA or DNA.

In certain aspects, the functional domain is a nuclease comprising a cleavage domain or a half-cleavage domain and the endogenous gene is inactivated by cleavage and wherein the first binding member comprises a NBD that binds to a first target nucleic acid sequence in the gene and the second binding member comprises a half-cleavage domain and the method comprises introducing a second first binding member comprising a NBD that binds to a second target nucleic acid sequence in the gene and a second binding member comprising a half-cleavage domain. In certain aspects, the first target nucleic acid sequence and the second target sequence are spaced apart in the gene and the two half-cleavage domains mediate a cleavage of the gene sequence at a location in between the first and second target nucleic acid sequences.

A method of introducing an exogenous nucleic acid into a region of interest in the genome of a cell is also provided. The method comprises:

introducing into the cell: the first binding member and the second binding member as disclosed herein, and the exogenous nucleic acid; or introducing into the cell: the first binding member and the second binding member as disclosed herein, and the exogenous nucleic acid, wherein the NBD of the polypeptide binds to the target nucleic acid sequence present adjacent the region of interest, wherein the cleavage domain or the half-cleavage domain introduces a cleavage in the region of interest and wherein the exogenous nucleic acid in integrated into the cleaved region of interest by homologous recombination.

In certain aspects, introducing the first binding member and the second biding member into the cell comprises contacting the cell with the first and second binding members in absence of a transfection agent, wherein the first and second binding members penetrate the cell membrane. In certain aspects, introducing the first and second binding members and the exogenous nucleic acid into the cell comprises contacting the cell with a composition comprising the first and second binding members associated with the exogenous nucleic acid, wherein the first and second binding members penetrate the cell membrane and transports the exogenous nucleic acid into the cell. Introducing may include administering the first and second binding members to a subject by e.g., parenteral administration. In certain aspects, the administering comprises intravenous, intramuscular, intrathecal, or subcutaneous administration. In certain aspects, the administering comprises direct injection into a site in a subject. In certain aspects, the administering comprises direct injection into a tumor.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: Reversibly Charged TALENS

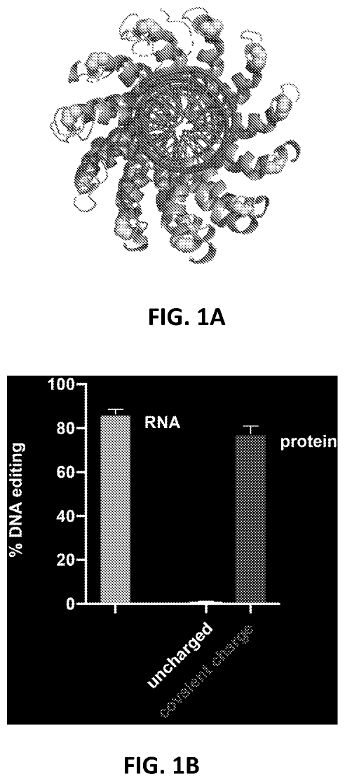

As a proof of concept, we delivered a TALEN pair targeting the AAVS1 safe harbor genomic locus using a method is adapted from Liu J., et al. (2014), PLoS ONE 9(1): e85755. Since each TALE repeat contains a single available cystine residue, we conjugated a cystine reactive moiety in each TALE repeat to an Arg₉ repeat peptide (FIG. 1A). After conjugation in basic conditions, the reaction was quenched, and K562 cells were treated with 10 nM TALEN-Arg₉ protein. After 4 hours, cells were treated DTT to release Arg₉ repeat peptide from the TALEN and editing efficiency was measured 24 hours later. Protein-mediated genome editing performed comparably to editing achieved by RNA transfection of the TALEN pair. FIG. 1B.

### Example 2: Cell Permeable Functional Domain

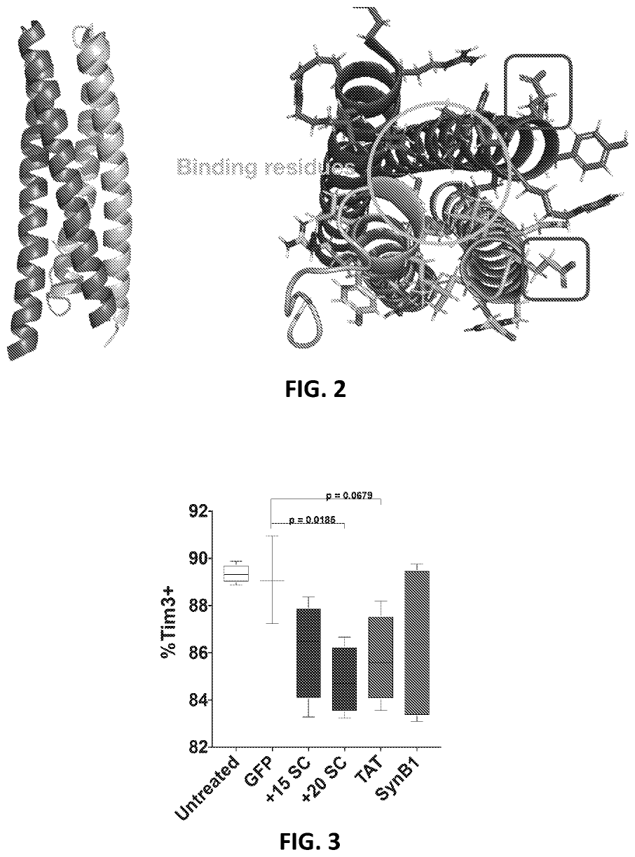

The Baker Lab recently reported a series of small obligate heterodimer proteins (Chen Z. et al., Nature 565, 106-111, 2019). The dimer interface is helix-like, with critical interactions between dimer partners occurring in the center, with non-interacting residues decorating the solvent-exposed dimer backbones. See FIG. 2. We rationally designed a series of dimer pairs where these solvent-exposed residues are mutated to charged amino acids (lysine or arginine). FIG. 2. Dimer pairs are referred to as 37A and 37B. The 37B designs are fused to a KRAB domain for testing in an epigenome editing assay.

As a pilot experiment for cell-penetrating activity of the 37B-KRAB fusion proteins, we synthesized the protein using an in vitro coupled transcription-translation system. The sequences of these two proteins are as follows:

37B-linker-KRAB-net15 “+15SC”
(SEQ ID NO: 158)
MKDKKLDKLLDKLEKILQKATKIIDKANKLLEKLRRSKRKKPKVVKTYVEL
LKRHEKAVKELLEIAKTHAKKVEGSGGGGGMDAKSLTAWSRTLVTFKDVFV
DFTREEWKLLDTAQQIVYRNVMLENYKNLVSLGYQLTKPDVILRLEKGEEP
37B-linker-KRAB-net20 “+20SC”
(SEQ ID NO: 159)
MKKDKKLDKLLDKLEKILQKATKIIDKANKLLEKLRRSKRKKPKVVKTYVE
LLKRHEKAVKELLEIAKTHAKKVEGKGSKGKGKGKMDAKSLTAWSRTLVTF
KDVFVDFTREEWKLLDTAQQIVYRNVMLENYKNLVSLGYQLTKPDVILRLE
KGEEP

The following constructs directly bind to promoter of TIM3 gene and served as positive controls:

>TAT-37B-linker-KRAB
(SEQ ID NO: 160)
MGRKKRRQRRRPPQDDKELDKLLDTLEKILQTATKIIDDANKLLEKLRRS
ERKDPKVVETYVELLKRHEKAVKELLEIAKTHAKKVEGSGGGGGMDAKSL
TAWSRTLVTFKDVFVDFTREEWKLLDTAQQIVYRNVMLENYKNLVSLGYQ
LTKPDVILRLEKGEEP
>SynB1-37B-linker-KRAB
(SEQ ID NO: 161)
MRGGRLSYSRRRFSTSTGRDDKELDKLLDTLEKILQTATKIIDDANKLLE
KLRRSERKDPKVVETYVELLKRHEKAVKELLEIAKTHAKKVEGSGGGGGM
DAKSLTAWSRTLVTFKDVFVDFTREEWKLLDTAQQIVYRNVMLENYKNLV
SLGYQLTKPDVILRLEKGEEP

Primary human T cells were transfected with the DNA binding domain targeting the TIM3 promoter fused to 37A, allowed to recover for 24 hours, then treated with the 37B-KRAB protein at ˜100 pM. Even with this small dose, we observe a statically significant reduction in TIM3 expression for the 37B-net20 charged KRAB construct, suggesting that these proteins are able to penetrate the cell, partner with the 37A DNA binding domain and nucleate repression at the TIM3 gene. FIG. 3.

For reasons of completeness, certain aspects of the polypeptides, composition, and methods of the present disclosure are set out in the following numbered clauses:

1. A polypeptide comprising a nucleic acid-binding domain comprising:
at least three repeat units comprising a 33-36 amino acid long sequence having at least 80% sequence identity to the amino acid sequence:

(SEQ ID NO: 1)
LTPDQ VVAIA SX¹²X¹³GG KQALE TVQRL LPVLC QDHG,

or
having the sequence of SEQ ID NO:1 with one or more conservative amino acid substitutions thereto; and
comprising at least one of the following amino acid substitutions relative to SEQ ID NO:1:

### D4K/R/H; S11K/R/H; Q23K/R/H; C30K/R/H; and D32K/R/H,

wherein X¹²X¹³ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent,
wherein when the repeat unit comprises the substitution D4K, X¹²X¹³ is not HN, YK or YG or wherein when the repeat unit comprises the substitution D4K, the repeat unit further comprises at least one of the following substitutions S11K/R/H; Q23K/R/H; C30K/R/H; and D32K/R/H,
wherein when the repeat unit comprises the substitution S11K, X₁₂X₁₃ is not RG or NI, or wherein when the repeat unit comprises the substitution S11K, the repeat unit further comprises at least one of the following substitutions D4K/R/H; Q23K/R/H; C30K/R/H; and D32K/R/H,
wherein when the repeat unit comprises the substitution Q23K, X₁₂X₁₃ is not SI, CI, or NN, wherein when the repeat unit comprises the substitution Q23R, X₁₂X₁₃ is not NG, or the repeat unit further comprises at least one of the following substitutions D4K/R/H; S11K/R/H; C30K/R/H; and D32K/R/H,
wherein when the repeat unit comprises the substitution C30R, X₁₂X₁₃ is not NS, HD, NI, NN, NH or NK, or the repeat unit further comprises at least one of the following substitutions D4K/R/H; S11K/R/H; Q23K/R/H; and D32K/R/H,
wherein when the repeat unit comprises the substitution D32H, X₁₂X₁₃ is not NG, or the repeat unit further comprises at least one of the following substitutions D4K/R/H; S11K/R/H; Q23K/R/H; and C30K/R/H, and
wherein the repeat unit has a net charge of at least +2.
2. The polypeptide of clause 1, wherein the 33-36 long amino acid sequence of the repeat unit has at least 80% sequence identity to the amino acid sequence set forth in one of SEQ ID NOs:17-26, wherein at least one of the amino acid residues at positions 4, 11, 23, and 32 has a positively charged side chain.
3. The polypeptide of clause 1 or 2, wherein the polypeptide is fused to a heterologous functional domain.
4. The polypeptide of clause 3, wherein the heterologous functional domain comprises an enzyme, a transcriptional activator, a transcriptional repressor, or a DNA nucleotide modifier.
5. The polypeptide of clause 4, wherein the enzyme is a nuclease, a DNA modifying protein, or a chromatin modifying protein.
6. The polypeptide of clause 5, wherein the nuclease is a cleavage domain or a half-cleavage domain.
7. The polypeptide of clause 6, the cleavage domain or half-cleavage domain comprises a type IIS restriction enzyme.
8. The polypeptide of clause 7, wherein the type IIS restriction enzyme comprises FokI or Bfil.
9. The polypeptide of clause 5, wherein the chromatin modifying protein is lysine-specific histone demethylase 1 (LSD1).
10. The polypeptide of clause 4, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
11. The polypeptide of clause 4, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
12. The polypeptide clause 4, wherein the DNA nucleotide modifier is adenosine deaminase.
13. A recombinant polypeptide comprising a nucleic acid binding domain (NBD) and a heterologous functional domain, the NBD comprising at least three repeat units (RUs) ordered from N-terminus to C-terminus of the NBD to specifically bind to a target nucleic acid, wherein each of the RUs comprises the sequence:
X_{1 to y}—X_{y+1}X_{y+2}—X_{(13 or 14)-(33 or 34 or 35)}, wherein
X_{1-y}, where y=10 or 11, is a chain of 10 or 11 contiguous amino acids,
X_{y+1}X_{y+2} is a diresidue present at positions 11 and 12 or 12 and 13,
X_{(13 or 14) to (33 or 34 or 35)} is a chain of 21, 22 or 23 contiguous amino acids, starting at position 13, when the diresidue is present at positions 11 and 12 or starting at position 14, when the diresidue is present at positions 11 and 12,
the net charge of each of the RUs is at least +2, and
the net charge of the polypeptide is at least +30.
14. The polypeptide of clause 13, wherein each RU independently comprises a 33-36 amino acid long sequence that is at least 80% identical to one of SEQ ID NOs: 27-88.
15. The polypeptide of clause 13, wherein each RU independently comprises a 33-36 amino acid long sequence that is at least 80% identical to one of SEQ ID NOs:89-121.
16. The polypeptide of clause 13, wherein each RU independently comprises a 33-36 amino acid long sequence that is at least 80% identical to one of SEQ ID NOs: 122-130.
17. The polypeptide of clause 13, wherein each RU independently comprises a 33-36 amino acid long sequence that is at least 80% identical to one of SEQ ID NOs:131-137.
18. The polypeptide of clause 13, wherein at least one RU comprises a 33-36 amino acid long sequence that is at least 80% identical to SEQ ID NO:138.
19. The polypeptide of clause 13, wherein at least one RU comprises a 33-36 amino acid long sequence that is at least 80% identical to SEQ ID NO:139.
20. The polypeptide of any one of clauses 13-19, wherein the heterologous functional domain comprises an enzyme, a transcriptional activator, a transcriptional repressor, or a DNA nucleotide modifier.
21. The polypeptide of clause 20, wherein the enzyme is a nuclease, a DNA modifying protein, or a chromatin modifying protein.
22. The polypeptide of clause 21, wherein the nuclease is a cleavage domain or a half-cleavage domain.
23. The polypeptide of clause 22, the cleavage domain or half-cleavage domain comprises a type IIS restriction enzyme.
24. The polypeptide of clause 23, wherein the type IIS restriction enzyme comprises FokI or Bfil.
25. The polypeptide of clause 21, wherein the chromatin modifying protein is lysine-specific histone demethylase 1 (LSD1).
26. The polypeptide of clause 20, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
27. The polypeptide of clause 20, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
28. The polypeptide clause 20, wherein the DNA nucleotide modifier is adenosine deaminase.
29. A first binding member of a heterodimer, wherein the first binding member comprises an amino acid sequence at least 75% identical to the amino acid sequence of SEQ ID NO:2 and comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H and wherein the first binding member binds to a second binding member of the heterodimer, wherein the second binding member comprises an amino acid sequence at least 75% identical to the amino acid sequence of SEQ ID NO:3.
30. The first binding member of clause 11, comprising at least three of the substitutions.
31. The first binding member of clause 11, comprising at least five of the substitutions.
32. The first binding member of clause 11, comprising at least eight of the substitutions.
33. The first binding member of any one of clauses 29-32, fused to a nucleic acid binding domain (NBD).
34. The first binding member of 33, wherein the NBD is fused to the N-terminus of the first binding member.
35. The first binding member of 33, wherein the NBD is fused to the C-terminus of the first binding member.
36. The first binding member of any one of clauses 33-35, wherein the NBD comprises a transcription activator-like effector (TALE), modular animal pathogen nucleic acid binding domain, zinc finger protein, or single-guide RNA.
37. The first binding member of any one of clauses 29-32, fused to a functional domain.
38. The first binding member of 37, wherein the functional domain is fused to the N-terminus of the first binding member.
39. The first binding member of 37, wherein the NBD is fused to the C-terminus of the first binding member.
40. The first binding member of any one of clauses 37-39, wherein the functional domain comprises an enzyme, a transcriptional activator, a transcriptional repressor, or a DNA nucleotide modifier.
41. The first binding member of clause 40, wherein the enzyme is a nuclease, a DNA modifying protein, or a chromatin modifying protein.
42. The first binding member of clause 41, wherein the nuclease is a cleavage domain or a half-cleavage domain.
43. The first binding member of clause 42, the cleavage domain or half-cleavage domain comprises a type IIS restriction enzyme.
44. The first binding member of clause 43, wherein the type IIS restriction enzyme comprises FokI or Bfil.
45. The first binding member of clause 41, wherein the chromatin modifying protein is lysine-specific histone demethylase 1 (LSD1).
46. The first binding member of clause 40, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
47. The first binding member of clause 40, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
48. The first binding member clause 40, wherein the DNA nucleotide modifier is adenosine deaminase.
49. A second binding member of a heterodimer, wherein the second binding member comprises an amino acid sequence at least 75% identical to the amino acid sequence of SEQ ID NO:3 and comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H and wherein the second binding member binds to a first binding member of the heterodimer, wherein the first binding member comprises an amino acid sequence at least 75% identical to the amino acid sequence of SEQ ID NO:2.
50. The second binding member of clause 49, comprising at least three of the substitutions.
51. The second binding member of clause 49, comprising at least five of the substitutions.
52. The second binding member of clause 49, comprising at least seven of the substitutions.
53. The second binding member of any one of clauses 49-52, fused to a nucleic acid binding domain (NBD).
54. The second binding member of 33, wherein the NBD is fused to the N-terminus of the first binding member.
55. The second binding member of 33, wherein the DBD is fused to the C-terminus of the first binding member.
56. The second binding member of any one of clauses 33-35, wherein the NBD comprises a transcription activator-like effector (TALE), modular animal pathogen nucleic acid binding domain, zinc finger protein, or single-guide RNA.
57. The second binding member of any one of clauses 49-52, fused to a functional domain.
58. The second binding member of 57, wherein the functional domain is fused to the N-terminus of the first binding member.
59. The second binding member of 57, wherein the NBD is fused to the C-terminus of the first binding member.
60. The second binding member of any one of clauses 57-59, wherein the functional domain comprises an enzyme, a transcriptional activator, a transcriptional repressor, or a DNA nucleotide modifier.
61. The second binding member of clause 60, wherein the enzyme is a nuclease, a DNA modifying protein, or a chromatin modifying protein.
62. The second binding member of clause 61, wherein the nuclease is a cleavage domain or a half-cleavage domain.
63. The second binding member of clause 62, the cleavage domain or half-cleavage domain comprises a type IIS restriction enzyme.
64. The second binding member of clause 63, wherein the type IIS restriction enzyme comprises FokI or Bfil.
65. The second binding member of clause 61, wherein the chromatin modifying protein is lysine-specific histone demethylase 1 (LSD1).
66. The second binding member of clause 60, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
67. The second binding member of clause 60, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
68. The second binding member clause 60, wherein the DNA nucleotide modifier is adenosine deaminase.
69. A heterodimer comprising the first binding member of any one of clauses 29-48 and the second binding member of any one of clauses 49-68.
70. The heterodimer of clause 69, wherein the first binding member is fused to a functional domain.
71. The heterodimer of clause 70, wherein the first binding member is fused to the N-terminus of the functional domain.
72. The heterodimer of clause 70 or 71, wherein the second binding member is fused to a DNA binding domain.
73. The heterodimer of clause 72, wherein the second binding member is fused to the C-terminus of the DNA binding domain.
74. The heterodimer of clause 69, wherein the second binding member is fused to a functional domain.
75. The heterodimer of clause 70, wherein the second binding member is fused to the N-terminus of the functional domain.
76. The heterodimer of clause 70 or 71, wherein the first binding member is fused to a DNA binding domain.
77. The heterodimer of clause 72, wherein the first binding member is fused to the C-terminus of the DNA binding domain.
78. The first binding member of any one of clauses 29-48, wherein the first binding member comprises a net charge of at least +15.
79. The second binding member of any one of clauses 49-68, wherein the second binding member comprises a net charge of at least +15.
80. The heterodimer of any one of clauses 69-77, wherein the first binding member and the second binding member each comprise a net charge of at least +15.
81. A pharmaceutical composition comprising the polypeptide of any of clauses 1-12, the recombinant polypeptide of any one of clauses 13-28, the first binding member of any one of clauses 29-48 and clause 78, the second binding member of any one of clauses 49-68 and clause
79, the first binding member and the second binding member of the heterodimer of any one of clauses 69-77 and clause 80; and a pharmaceutically acceptable excipient.
82. A nucleic acid encoding the polypeptide of any one of clauses 1-12.
83. A nucleic acid encoding the recombinant polypeptide of any one of clauses 13-28.
84. A nucleic acid encoding the first binding member of any one of clauses 29-48 and 78.
85. A nucleic acid encoding the second binding member of any one of clauses 49-68 and 79.
86. One or more nucleic acids encoding the heterodimer of any one of clauses 69-77 and 80.
87. A method of modulating expression of an endogenous gene in a cell, the method comprising:

contacting the cell with the polypeptide of any one of clauses 3 or clauses 13-19,

wherein the polypeptide penetrates the cell membrane and wherein the NBD of the polypeptide binds to a target nucleic acid sequence present in the endogenous gene and the heterologous functional domain modulates expression of the endogenous gene.

88. The method of clause 87, wherein the nucleic acid is a ribonucleic acid (RNA).
89. The method of clause 87, wherein the nucleic acid is a deoxyribonucleic acid (DNA).
90. The method of any of clauses 87-89, wherein the functional domain is a transcriptional activator and the target nucleic acid sequence is present in an expression control region of the gene, wherein the polypeptide increases expression of the gene.
91. The method of clause 90, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
92. The method of any of clauses 87-89, wherein the functional domain is a transcriptional repressor and the target nucleic acid sequence is present in an expression control region of the gene, wherein the polypeptide decreases expression of the gene.
93. The method of clause 92, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
94. The method of any of clauses 87-93, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAl gene, a TTR gene, a NR3Cl gene, a CD52 gene, an erythroid specific enhancer of the ECLllA gene, a CELE gene, a TGFERl gene, a SERPINAl gene, a HEV genomic DNA in infected cells, a CEP290 gene, a DMD gene, a CFTR gene, or an IL2RG gene.
95. The method of any of clauses 90-94, wherein the expression control region of the gene comprises a promoter region of the gene.
96. The method of any of clauses 87-89, wherein the functional domain is a nuclease comprising a cleavage domain or a half-cleavage domain and the endogenous gene is inactivated by cleavage.
97. The method of clause 96, wherein the polypeptide is a first polypeptide that binds to a first target nucleic acid sequence in the gene and comprises a half-cleavage domain and the method comprises introducing a second polypeptide that binds to a second target nucleic acid sequence in the gene and comprises a half-cleavage domain.
98. The method of clause 97, wherein the first target nucleic acid sequence and the second target sequence are spaced apart in the gene and the two half-cleavage domains mediate a cleavage of the gene sequence at a location in between the first and second target nucleic acid sequences.
99. The method of any of clauses 96-98, wherein the cleavage domain or the cleavage half domain comprises FokI or Bfil.
100. The method of clause 82 or 83, wherein FokI has a sequence of SEQ ID NO: 11.
101. The method of clause 96, wherein the cleavage domain comprises a meganuclease.
102. The method of any of clauses 96-101, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAl gene, a TTR gene, a NR3Cl gene, a CD52 gene, an erythroid specific enhancer of the ECLllA gene, a CELE gene, a TGFERl gene, a SERPINAl gene, a HEV genomic DNA in infected cells, a CEP290 gene, a DMD gene, a CFTR gene, or an IL2RG gene.
103. A method of introducing an exogenous nucleic acid into a region of interest in the genome of a cell, the method comprising: introducing into the cell: the polypeptide of any one of clauses 6-8 or clauses 22-24, wherein the NBD of the polypeptide binds to the target nucleic acid sequence present adjacent the region of interest, and the exogenous nucleic acid, wherein the cleavage domain or the half-cleavage domain introduces a cleavage in the region of interest and wherein the exogenous nucleic acid in integrated into the cleaved region of interest by homologous recombination.
104. The method of clause 103, wherein introducing the polypeptide into the cell comprises contacting the cell with the polypeptide in absence of a transfection agent, wherein the polypeptide penetrates the cell membrane.
105. The method of clause 103, wherein introducing the polypeptide and the exogenous nucleic acid into the cell comprises contacting the cell with a composition comprising the polypeptide associated with the exogenous nucleic acid, wherein the polypeptide penetrates the cell membrane and transports the exogenous nucleic acid into the cell.
106. The method of any of clauses 87-105, wherein the cell is an animal cell or plant cell.
107. The method of any of clauses 87-105, wherein the cell is a human cell.
108. The method of any of clauses 87-107, wherein the cell is an ex vivo cell.
109. The method of any of clauses 67-101, wherein the introducing comprises administering the polypeptide to a subject.
110. The method of any of clause 109, wherein the administering comprises parenteral administration.
111. The method of any of clause 109, wherein the administering comprises intravenous, intramuscular, intrathecal, or subcutaneous administration.
112. The method of any of clause 109, wherein the administering comprises direct injection into a site in a subject.
113. The method of any of clause 109, wherein the administering comprises direct injection into a tumor.
114. A method of modulating expression of an endogenous gene in a cell, the method comprising: introducing into the cell the first binding member of any one of clauses 33-36 and the second binding member of any one of clauses 57-68, wherein at least one of the first and second binding members penetrates the cell membrane and wherein the NBD binds to a target nucleic acid sequence present in the endogenous gene and the heterologous functional domain modulates expression of the endogenous gene; or introducing into the cell the first binding member of any one of clauses 37-48 and the second binding member of any one of clauses 53-56, wherein at least one of the first and second binding members penetrates the cell membrane and wherein the NBD binds to a target nucleic acid sequence present in the endogenous gene and the heterologous functional domain modulates expression of the endogenous gene; or the heterodimer of any one of clauses 70-77, wherein at least the first and second binding members penetrates the cell membrane and wherein the NBD binds to a target nucleic acid sequence present in the endogenous gene and the heterologous functional domain modulates expression of the endogenous gene.
115. The method of clause 114, wherein introducing into the cell the first and second binding members comprises contacting the cell with the first and second binding members.
116. The method of clause 114, wherein introducing into the cell the first and second binding members comprises contacting the cell with the first binding member and introducing into the cell a nucleic acid encoding the second binding member.
117. The method of clause 114, wherein introducing into the cell the first and second binding members comprises contacting the cell with the second binding member and introducing into the cell a nucleic acid encoding the first binding member.
118. The method of any one of clauses 113-117, wherein the nucleic acid is a ribonucleic acid (RNA).
119. The method of any one of clauses 113-117, wherein the nucleic acid is a deoxyribonucleic acid (DNA).
120. The method of any of clauses 113-119, wherein the functional domain is a transcriptional activator and the target nucleic acid sequence is present in an expression control region of the gene, wherein the method increases expression of the gene.
121. The method of clause 120, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).
122. The method of any of clauses 113-119, wherein the functional domain is a transcriptional repressor and the target nucleic acid sequence is present in an expression control region of the gene, wherein the method decreases expression of the gene.
123. The method of clause 122, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.
124. The method of any of clauses 113-123, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3Cl gene, a CD52 gene, an erythroid specific enhancer of the BCL11A gene, a CELE gene, a TGFERl gene, a SERPINAl gene, a HEV genomic DNA in infected cells, a CEP290 gene, a DMD gene, a CFTR gene, or an IL2RG gene.
125. The method of any of clauses 122-124, wherein the expression control region of the gene comprises a promoter region of the gene.
126. The method of any of clauses 113-119, wherein the functional domain is a nuclease comprising a cleavage domain or a half-cleavage domain and the endogenous gene is inactivated by cleavage.
127. The method of clause 126, wherein the first binding member comprises a NBD that binds to a first target nucleic acid sequence in the gene and the second binding member comprises a half-cleavage domain and the method comprises introducing a second first binding member comprising a NBD that binds to a second target nucleic acid sequence in the gene and a second binding member comprising a half-cleavage domain.
128. The method of clause 127, wherein the first target nucleic acid sequence and the second target sequence are spaced apart in the gene and the two half-cleavage domains mediate a cleavage of the gene sequence at a location in between the first and second target nucleic acid sequences.
129. The method of any of clauses 126-128, wherein the cleavage domain or the cleavage half domain comprises FokI or Bfil.
130. The method of clause 129, wherein FokI has a sequence of SEQ ID NO: 11.
131. The method of clause 126, wherein the cleavage domain comprises a meganuclease.
132. The method of any of clauses 126-131, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3Cl gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.
133. A method of introducing an exogenous nucleic acid into a region of interest in the genome of a cell, the method comprising: introducing into the cell: the first binding member of any one of clauses 33-36 and the second binding member of any one of clauses 62-64, and the exogenous nucleic acid; or introducing into the cell: the first binding member of any one of clauses 42-44 and the second binding member of any one of clauses 53-57, and the exogenous nucleic acid, wherein the NBD of the polypeptide binds to the target nucleic acid sequence present adjacent the region of interest, wherein the cleavage domain or the half-cleavage domain introduces a cleavage in the region of interest and wherein the exogenous nucleic acid in integrated into the cleaved region of interest by homologous recombination.
134. The method of clause 133, wherein introducing the first binding member and the second biding member into the cell comprises contacting the cell with the first and second binding members in absence of a transfection agent, wherein the first and second binding members penetrate the cell membrane.
135. The method of clause 134, wherein introducing the first and second binding members and the exogenous nucleic acid into the cell comprises contacting the cell with a composition comprising the first and second binding members associated with the exogenous nucleic acid, wherein the first and second binding members penetrate the cell membrane and transports the exogenous nucleic acid into the cell.
136. The method of any of clauses 114-135, wherein the cell is an animal cell or plant cell.
137. The method of any of clauses 114-135, wherein the cell is a human cell.
138. The method of any of clauses 114-135, wherein the cell is an ex vivo cell.
139. The method of any of clauses 114-135, wherein the introducing comprises administering the first and second binding members to a subject.
140. The method of any of clause 139, wherein the administering comprises parenteral administration.
141. The method of any of clause 139, wherein the administering comprises intravenous, intramuscular, intrathecal, or subcutaneous administration.
142. The method of any of clause 139, wherein the administering comprises direct injection into a site in a subject.
143. The method of any of clause 139, wherein the administering comprises direct injection into a tumor.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1.-143. (canceled)

144. A polypeptide comprising a nucleic acid-binding domain comprising:
at least three repeat units comprising a 33-36 amino acid long sequence having at least 80% sequence identity to the amino acid sequence:
(SEQ ID NO: 1)
LTPDQ VVAIA SX¹²X¹³GG KQALE TVQRL LPVLC QDHG,
having the sequence of SEQ ID NO:1 with one or more conservative amino acid substitutions thereto; and
comprising at least one of the following amino acid substitutions relative to SEQ ID NO:1:
D4K/R/H; S11K/R/H; Q23K/R/H; C30K/R/H; and D32K/R/H,
wherein X¹²X¹³ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent,
wherein when the repeat unit comprises the substitution D4K, X¹²X¹³ is not HN, YK or YG or wherein when the repeat unit comprises the substitution D4K, the repeat unit further comprises at least one of the following substitutions S11K/R/H; Q23K/R/H; C30K/R/H; and D32K/R/H,
wherein when the repeat unit comprises the substitution S11K, X₁₂X₁₃ is not RG or NI, or wherein when the repeat unit comprises the substitution S11K, the repeat unit further comprises at least one of the following substitutions D4K/R/H; Q23K/R/H; C30K/R/H; and D32K/R/H,
wherein when the repeat unit comprises the substitution Q23K, X₁₂X₁₃ is not SI, CI, or NN, wherein when the repeat unit comprises the substitution Q23R, X₁₂X₁₃ is not NG, or the repeat unit further comprises at least one of the following substitutions D4K/R/H; S11K/R/H; C30K/R/H; and D32K/R/H,
wherein when the repeat unit comprises the substitution C30R, X₁₂X₁₃ is not NS, HD, NI, NN, NH or NK, or the repeat unit further comprises at least one of the following substitutions D4K/R/H; S11K/R/H; Q23K/R/H; and D32K/R/H,
wherein when the repeat unit comprises the substitution D32H, X₁₂X₁₃ is not NG, or the repeat unit further comprises at least one of the following substitutions D4K/R/H; S11K/R/H; Q23K/R/H; and C30K/R/H, and
wherein the repeat unit has a net charge of at least +2.

145. The polypeptide of claim 144, wherein the 33-36 long amino acid sequence of the repeat unit has at least 80% sequence identity to the amino acid sequence:
i.
(SEQ ID NO: 17)
LTPKQ VVAIA SX₁₂X₁₃GG KQALE TVQRL LPVLC QDHG;
ii.
(SEQ ID NO: 18)
LTPRQ VVAIA SX₁₂X₁₃GG KQALE TVQRL LPVLC QDHG;
iii.
(SEQ ID NO: 19)
LTPDQ VVAIA KX₁₂X₁₃GG KQALE TVQRL LPVLC QDHG;
iv.
(SEQ ID NO: 20)
LTPDQ VVAIA RX₁₂X₁₃GG KQALE TVQRL LPVLC QDHG;
v.
(SEQ ID NO: 21)
LTPDQ VVAIA SX₁₂X₁₃GG KQALE TVKRL LPVLC QDHG;
vi.
(SEQ ID NO: 22)
LTPDQ VVAIA SX₁₂X₁₃GG KQALE TVRRL LPVLC QDHG;
vii.
(SEQ ID NO: 23)
LTPDQ VVAIA SX₁₂X₁₃GG KQALE TVQRL LPVLK QDHG;
viii.
(SEQ ID NO: 24)
LTPDQ VVAIA SX₁₂X₁₃GG KQALE TVQRL LPVLR QDHG;
ix.
(SEQ ID NO: 25)
LTPDQ VVAIA SX₁₂X₁₃GG KQALE TVQRL LPVLC QKHG;
or
x.
(SEQ ID NO: 26)
LTPDQ VVAIA SX₁₂X₁₃GG KQALE TVQRL LPVLC QRHG,
wherein at least one of the amino acid residues at positions 4, 11, 23, and 32 has a positively charged side chain.

146. The polypeptide of claim 144, wherein the polypeptide is fused to a heterologous functional domain.

147. The polypeptide of claim 146, wherein the heterologous functional domain comprises an enzyme, a transcriptional activator, a transcriptional repressor, or a DNA nucleotide modifier.

148. The polypeptide of claim 147, wherein the enzyme is a nuclease, a DNA modifying protein, or a chromatin modifying protein.

149. The polypeptide of claim 147, wherein the transcriptional activator comprises VP16, VP64, p65, p300 catalytic domain, TET1 catalytic domain, TDG, Ldb1 self-associated domain, SAM activator (VP64, p65, HSF1), or VPR (VP64, p65, Rta).

150. The polypeptide of claim 147, wherein the transcriptional repressor comprises KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2.

151. The polypeptide of claim 147, wherein the DNA nucleotide modifier is adenosine deaminase.

152. A pharmaceutical composition:
comprising the polypeptide of claim 144; and
a pharmaceutically acceptable excipient.

153. A nucleic acid encoding the polypeptide of claim 144.

154. A method of modulating expression of an endogenous gene in a cell, the method comprising:
contacting the cell with the polypeptide of claim 146,
wherein the polypeptide penetrates the cell membrane and wherein the NBD of the polypeptide binds to a target nucleic acid sequence present in the endogenous gene and the heterologous functional domain modulates expression of the endogenous gene.

155. The method of claim 154, wherein the functional domain is a transcriptional activator and the target nucleic acid sequence is present in an expression control region of the gene, wherein the polypeptide increases expression of the gene.

156. The method of claim 154, wherein the functional domain is a transcriptional repressor and the target nucleic acid sequence is present in an expression control region of the gene, wherein the polypeptide decreases expression of the gene.

157. The method of claim 154, wherein the gene is a PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEA1 gene, a TTR gene, a NR3Cl gene, a CD52 gene, an erythroid specific enhancer of the BCL11A gene, a CELE gene, a TGFER1 gene, a SERPINA1 gene, a HEV genomic DNA in infected cells, a CEP290 gene, a DMD gene, a CFTR gene, or an IL2RG gene.

158. A first binding member of a heterodimer, wherein the first binding member comprises an amino acid sequence at least 75% identical to the amino acid sequence of SEQ ID NO:2 and comprises at least one of the following substitutions relative to the amino acid sequence of SEQ ID NO:2: D3K/R/H; E4K/R/H; T11K/R/H; D24K/R/H; D32K/R/H; S35K/R/H; E39K/R/H; D40K/R/H; E41K/R/H; D45K/R/H; D48K/R/H; L49K/R/H; T59K/R/H; and D66K/R/H and wherein the first binding member binds to a second binding member of the heterodimer, wherein the second binding member comprises an amino acid sequence at least 75% identical to the amino acid sequence of SEQ ID NO:3.

159. The first binding member of claim 158, comprising at least three of the substitutions.

160. The first binding member of claim 158, fused to a nucleic acid binding domain (NBD).

161. A second binding member of a heterodimer, wherein the second binding member comprises an amino acid sequence at least 75% identical to the amino acid sequence of SEQ ID NO:3 and comprises at least one of the following substitutions relative to the amino acid sequence of SEQ ID NO:3: D2K/R/H; D3K/R/H; E5K/R/H; T12K/R/H; T19K/R/H; D26K/R/H; E38K/R/H; D41K/R/H; E46K/R/H; E56K/R/H; E61K/R/H; T68K/R/H; and E74K/R/H and wherein the second binding member binds to a first binding member of the heterodimer, wherein the first binding member comprises an amino acid sequence at least 75% identical to the amino acid sequence of SEQ ID NO:2.

162. The second binding member of claim 161, comprising at least three of the substitutions.

163. The second binding member of claim 161, fused to a functional domain.
